(19) United States
(12) Patent Application Publication
(10) Pub. No.: US 2020/0113476 A1
(43) Pub. Date: Apr. 16, 2020

(54) METHOD FOR PRECISION CANCER TREATMENT BY IDENTIFYING DRUG RESISTANCE

(71) Applicant: Lan Jiang, DALLAS, TX (US)

(72) Inventor: Lan Jiang, DALLAS, TX (US)

(21) Appl. No.: 16/708,857

(22) Filed: Dec. 10, 2019

Related U.S. Application Data
(63) Continuation-in-part of application No. 15/275,897, filed on Sep. 26, 2016.
(60) Provisional application No. 62/233,682, filed on Sep. 28, 2015.

(51) Int. Cl. A61B 5/055; G06T 7/00; G06T 7/33; G06T 7/35; G16H 50/30; G16H 30/20; G06F 17/18

(52) U.S. Cl. CPC A61B 5/055; G06T 7/0014; G06T 7/337; G06T 7/35; G16H 10/40; G16H 50/30; G16H 30/20; G06F 17/18; G06T 7/97

(57) ABSTRACT

A method for precision cancer treatment by identifying drug resistance is provided. In some embodiments, the method may include: detecting tumor oxygenated perfusion by having a patient breathe air to acquire MRI baseline data; inhalation of hyperoxia gas to generate higher than baseline HbO₂ blood circulating in body to acquire MRI enhanced data; the region-of-interest (ROI), which in this case is a tumor volume (V₀), and which may be performed by volume contour tracing/region-of-interest (ROI) analysis and 3D tumor volumetry methods; calculating voxel's enhanced signal intensity (ΔSI); calculating tumor oxygenated perfusion percentage (OPP %); selecting different threshold and calculating maps such as a reconstruction OPP % pseudo color map; calculating tumor volume change ratio (Vt %); overlaying reconstruction OPP % pseudo color map to original images for visualizing tumor response data; drawing or plotting the OPP % and Vt % on a cancer treatment response information diagram, and identifying the type of drug resistance, classifying the drug resistance being caused by poor drug distribution factor or cells-specific factor based on pooled collected data.

### CROSS REFERENCE TO RELATED APPLICATIONS

This application is a continuation-in-part of U.S. Non-Provisional application Ser. No. 15/275,897, filed on Sep. 26, 2016, entitled “CANCER THERAPEUTIC WINDOW EVALUATION METHOD”, which claims the benefit of U.S. Provisional Application No. 62/233,682, filed on Sep. 28, 2015, entitled “CANCER TREATMENT EVALUATION METHOD”, the entire disclosures of which are incorporated by reference herein.

### FIELD OF THE INVENTION

This patent specification relates to the field of identification methods of tumor drug resistance. More specifically, a non-invasive method for identifying drug resistance in cancer treatment, this patent specification relates to computer implemented method of identifying drug resistance in solid cancer systemic therapies for improving cancer treatment outcomes.

### BACKGROUND

Although there are multiple therapeutic modalities (Chemotherapy, Radiotherapy, Immunotherapy, Molecular Targeted Therapy, etc.) available for cancer treatment in the clinical setting, clinicians still face the challenge of selecting the right therapeutic approach for each patient and balancing relative benefit with risk to achieve the most successful outcome. Because the vascular system of tumor plays a role of key importance during tumor growth, metastasis, and treatment. Tumor vascular system usually demonstrated inefficient in blood flow, oxygen and nutrition delivery comparing with normal tissue, which may directly cause the inefficient effectiveness in systemic therapies. For example, the poor microcirculatory perfusion regions of tumor can cause suboptimal the ability of drug/agent distribution in systemic therapy inside tumor which may lead to treatment failure in systemic therapies (Chemotherapy, Targeted therapy, Immunotherapy, Gene therapy, and Photodynamic therapy, etc.). Meanwhile, the tumor microcirculatory perfusion can be longitudinally changed with tumor shrinkage during treatment course, which also may cause huge variation in outcome. Because of the high heterogeneity of microcirculatory perfusion and oxygenation level both inter- and intra-tumor, it is one reason that the same stage patients with the same treatment can vary in outcome among patients. In addition, tumor cells may have vastly different responses to drugs/agents in systemic therapies due to difference in cancer cell genomic information, which may cause treatment failure too. Numerous studies have shown that the treatment resistance may be divided into two broad categories: cells-specific factors and pharmacological/physiological factors. Different types of resistance may require different treatment strategies to overcome resistance disorders in the clinical setting.

Timely monitoring and identifying the type of drug resistance of tumor in systemic treatment will benefit to developing or adjusting the optimal treatment plan during treatment course. It is of significance in reducing ineffective treatment or even ineffective over-treatment in the clinical setting. Unfortunately, the research shown that majority of human tumors are inefficient microcirculation which means the necessity and importance of identifying drug resistance in systemic therapies. For example, clinical statistics report that almost 70% of breast cancer patients may not respond to chemotherapy. Some of these may be caused by cell-specific factors, some of which may be due to low drug distribution/concentration due to poor microcirculation. However, although clinicians know that only 30% of patients will be completely or partially clinical response, all cancer patients may still have to undergo a chemotherapy regimen as a routine procedure without the information of tumor drug resistance. It may lead to clinically ineffective treatment, even ineffective overtreatment, which can damage the patient's health and waste time. Currently, there are no techniques or methods available to accurately identify the type of drug resistance during treatment.

Therefore, a need exists for novel methods of precision medicine which are able to provide the individualization of each patient's treatment for improving efficiency, which offers the ability of matching the right treatments to the right patients at the right time point to improve patient outcomes and quality of life. There also exists a need for novel method for identifying drug resistance as routine for reducing both exposure to ineffective therapies and the cost of cancer care. There is a further need for novel cancer technique platform which are able to visually aid in identifying, tracking, evaluating, and optimizing cancer therapy for customized evidence-based cancer treatment. There exists a need for novel technologic method that can help to early identify the type of drug resistance during the course of treatment or even before treatment. There exists a need for novel method that can provide treatment information of different therapeutic modalities on one platform for sharing with clinicians having different therapeutic backgrounds, comprehensively analyzing treatment, and searching the best therapeutic strategy. Finally, there exists a need for novel cancer therapeutic platform that provide the ability of real-time monitoring therapeutic previous response and future possible response information in adjusting and optimizing of current treatment plan during treatment course for achieving precision cancer treatment in clinical setting.

### BRIEF SUMMARY OF THE INVENTION

The invention is used in the treatment of cancer to identify drug resistance and to provide clinicians with evidence for optimizing cancer treatment plan during the course of treatment. The invention may include six innovative technology aspects. In some embodiments, the invention may include:

1. Using endogenous contrast agent dHbO₂ and applying special imaging protocol and T2 weighted MRI technique to monitor tumor therapeutic response during treatment course. In preferred embodiments, oxygenated perfusion percentage data OPP % is to use threshold technique in processing dynamic contrast enhancement T2 weighted MRI data for quantitatively measuring patient tumor microcirculation during the course of treatment or before treatment. Current clinical diagnostic imaging modalities which use extrinsic contrast agents may have a limitation to monitor therapeutic response information during cancer treatment course due to change of vascular permeability. Many cancer treatments may often lead to significant change of permeability. In some embodiments, an imaging protocol and technique of the present invention may use endogenous contrast agent dHbO₂ for non-invasively imaging tumor physiologic information (oxygenated perfusion) because oxygen in the blood can rapidly diffuse and exchange across the vessel wall independent of vascular permeability. The high-enhanced signal region represents a fresh oxygenated blood flow region and a good microcirculation region. Although such imaging protocols and techniques have been reported, a novel aspect of the present invention is that it is the first to enable the identification of the type of drug resistance in the cancer systemic treatment by novel technical methods.

2. Quantitatively analyzing tumor microcirculation. Although tumor microcirculation is a very important physiologic factors, to non-invasively evaluate tumor microcirculation in clinical routine remains a challenge. For dynamic contrast enhancement signals processing model, average of enhanced signal of all tumor region and its curve are often used to represent enhanced results which is not enough to evaluate whole tumor microcirculation. In preferred embodiments, the relative signal enhancement signal may be processed on voxel-by-voxel basis. An enhanced threshold may be set up to classify the level of fresh oxygenated perfusion inside tumor and all voxels of tumor which are more than threshold will be counted. Finally, the tumor microcirculation may be quantitatively evaluated as the percentage of tumor fresh oxygenated perfusion region. The higher the percentage of oxygenated perfusion region, the higher amount of fresh blood that flows in, and the better the microcirculation of the tumor, which may be related to possible future treatment outcomes. Medical research demonstrated that tumor microcirculation, as prognostic information, is associated with the effects of systemic therapy and radiotherapy.

3. Two parameters of the previous response and possible future response were used as a tumor treatment response information point. Changes in tumor volume are often used to assess treatment outcomes. However, tumor volumes in response to effective treatment are significantly delayed by days or weeks. The tumor volume information only reflects a result of previous treatment when measuring tumor during treatment course. The clinician wants to know both of this information to understand the previous treatment response of the tumor and the possible outcomes in order to make adjustment of treatment plan in time. Changes in tumor volume and tumor microcirculation may constitute a point in a two-dimensional coordinate system that represents previous treatment outcomes and possible future treatment outcomes. Visualization of measurement points (two-dimensional tumor response information) may be used to help track and identify different treatment resistances, thereby optimizing treatment strategies and reducing ineffective treatment.

4. Design a specific infographic and establish a unified standard for assessing cancer treatment information. It is not convenient to review and share all of the patient's previous treatment results with other clinicians. In order to easily understand and analyze treatment information by clinicians who have different treatment backgrounds, all treatment information may need to be aggregated in one diagram of infographic with uniform criteria. In the present invention, a novel specific cancer treatment response information diagram is designed for displaying all treatment response information points on one map. It may be used to help clinicians master the progress of treatment of tumors, optimize treatment plans in time, and reduce ineffective treatment. The results of systemic therapy and radiotherapy are shown in two symmetric coordinate systems respectively, which may be used to help clinicians verify the effects of different treatments. The relative value of the parameters as a uniform standard may be suitable for analyzing all solid tumor cases on a single diagram.

5. Develop an identification method for detecting the type of drug resistance. Clinical studies have shown that drug resistance in systemic therapy can be divided into two broad categories: cell-specific factors and pharmacological/physiological factors. The different type of drug resistance may need to take different treatment strategies to overcome their barriers. Timely identification of the type of resistance is extremely important for Precision Medicine in Cancer Treatment. Medical research found that majority of human tumors represent inefficient microcirculation. Drug resistance caused by low drug distribution/concentration (poor microcirculation), as one of the pharmacological/physiological categories, may be common in cancer treatment. Meanwhile, it has been reported that cancer cell resistance may lead to failure of targeted therapy due to mutations in targeted cancer genes. Identifying the type of treatment resistance from cell-specific factors is extremely important for targeted therapies. Determining the type of treatment resistance as early as possible will give patients and clinicians the opportunity to adjust treatment strategies in a timely manner during the treatment process. Currently, there are no clinically available methods for distinguishing types of resistance. In the present invention, the type of therapeutic resistance is able to be determined during treatment or even prior to treatment, which can greatly improve current cancer treatment techniques.

6. A computer implemented identification method for clinical application. Herein, the computer software system is configured to process MRI raw data, evaluate tumor microcirculation, generate tumor oxygenated perfusion distribution map, visualize treatment responding information on the infographic, assess patients' therapeutic information, identify the type of drug resistance. Also, the software system may be configured to run on different operation system platform and mobile devices in processing, recording, visualizing and sharing with clinicians for improving cancer treatment efficacy and reducing ineffective treatment.

The present invention may include six innovative aspects that perform the following functions: analyzing MRI raw data for detecting tumor physiological characteristics without the influence of vascular permeability, quantitatively assessing the ability of fresh oxidative blood through the tumor, integrating the previous therapeutic response and possible future outcomes into a therapeutic response information point, designing a specific infographic to visualize cancer treatment information points, establishing uniform criteria to visualize and compare all measured response information points, establishing clinical applicable method for identifying cancer drug resistances, and development of software for computerizing all the functions of the present invention on different operating system platforms.

The present invention provides a novel method for a clinician to timely identify the type of drug resistance during cancer treatment. The clinical significance of the present invention is to provide a novel clinically applicable technique and method in clinical routine for identifying drug resistance in a timely manner and assisting clinicians in conducting evidence-based cancer treatment. Cancer precision medicine is a method of providing the most suitable treatment method according to the characteristics of cancer. The present invention will provide a powerful tool for clinicians to monitor the characteristics of ineffective cancer treatment and monitor the artificial modification of the tumor environment for the best treatment conditions in real time. It will make clinical cancer treatments more controllable and efficient. Precision medicine in cancer treatment is expected to become a mainstream medicine in the near future, the present invention will play a very important role in precision cancer treatment.

### BRIEF DESCRIPTION OF THE DRAWINGS

Some embodiments of the present invention are illustrated as an example and are not limited by the figures of the accompanying drawings, in which like references may indicate similar elements and in which:

FIG. 1 depicts a block diagram of an example of a method for precision cancer treatment by identifying drug resistance according to various embodiments described herein.

FIG. 2 illustrates an example of a cancer treatment response information diagram according to various embodiments described herein.

FIG. 3A illustrates an example of breast cancer according to various embodiments described herein.

FIG. 3B illustrates an example cross sectional MR image of breast cancer according to various embodiments described herein.

FIG. 4A shows an example of a breast cancer tumor size measurement prior to an ineffective chemotherapy treatment situation. Shaded area indicates that the area is greater than the threshold, and generating a gray map optionally uses only a single threshold.

FIG. 4B shows an example of a breast cancer tumor size measurement of the breast cancer tumor of FIG. 4A after a first an ineffective chemotherapy treatment course where OPP % is low and the tumor volume reduces small during treatment. Shaded area indicates that the area is greater than the threshold, and generating a gray map optionally uses only a single threshold.

FIG. 4C shows an example of a breast cancer tumor size measurement of the breast cancer tumor of FIG. 4A after a second an ineffective chemotherapy treatment course where OPP % is low and the tumor volume reduces small during treatment. Shaded area indicates that the area is greater than the threshold, and generating a gray map optionally uses only a single threshold.

FIG. 4D shows an example of a breast cancer tumor size measurement prior to an effective chemotherapy treatment situation. Shaded area indicates that the area is greater than the threshold, and generating a gray map optionally uses only a single threshold.

FIG. 4E shows an example of a breast cancer tumor size measurement of the breast cancer tumor of FIG. 4D after a first effective chemotherapy treatment course where OPP % is high and tumor volume largely decreases during treatment. Shaded area indicates that the area is greater than the threshold, and generating a gray map optionally uses only a single threshold.

FIG. 4F shows an example of a breast cancer tumor size measurement of the breast cancer tumor of FIG. 4D after a second effective chemotherapy treatment course where OPP % is high and tumor volume largely decreases during treatment. Shaded area indicates that the area is greater than the threshold, and generating a gray map optionally uses only a single threshold.

FIG. 5 shows an example (FIGS. 4A-4C) of a breast cancer treatment response information diagram which describes an ineffective chemotherapy, treatment resistance caused by inefficient drug distribution factor, cancer treatment according to various embodiments described herein.

FIG. 6 shows an example (FIGS. 4D-4F) of a cancer treatment response information diagram which describes an effective chemotherapy treatment according to various embodiments described herein.

FIG. 7 shows an example of a cancer treatment response information diagram which describes an ineffective chemotherapy or targeted therapy. The drug resistance is caused by cells-specific factors according to various embodiments described herein.

FIG. 8 shows an example of a cancer treatment response information diagram which describes an effective chemo-radiotherapy combination treatment according to various embodiments described herein.

FIG. 9 illustrates an example construction of a cancer treatment response information diagram according to various embodiments described herein.

FIG. 10 depicts an example of a block diagram of a server which may be used to perform one or more steps of the computer implemented a method for identifying the type of drug resistance in cancer treatment according to various embodiments described herein.

FIG. 11 illustrates an example of a block diagram of an electronic device which may be used to perform one or more steps of the computer implemented identification method and to generate a cancer treatment response information diagram according to various embodiments described herein.

FIG. 12 shows an illustrative example of some of the components and computer implemented methods which may be found in a cancer therapy treatment resistance identification system according to various embodiments described herein.

FIG. 13 depicts a block diagram illustrating some applications of a cancer therapy treatment resistance identification system which may function as software rules engines according to various embodiments described herein.

FIG. 14 illustrates a block diagram of an example of a method for generating an estimation of how to identify the type of therapy resistance of a cancer of a particular patient according to various embodiments described herein.

FIG. 15A shows a table providing some example critical values for evaluating tumor low drug distribution as a factor for determining resistance of a patient's cancer according to various embodiments described herein.

FIG. 15B shows a table providing some example critical values for determining resistance of a patient's cancer to anti-angiogenic therapy according to various embodiments described herein.

FIG. 15C shows a table providing some example critical values for evaluating tumor cells-specific factors for determining resistance of a patient's cancer according to various embodiments described herein.

FIG. 16 illustrates a block diagram of an example of a method for precision cancer treatment by identifying drug resistance according to various embodiments described herein.

### DETAILED DESCRIPTION OF THE INVENTION

The terminology used herein is for the purpose of describing particular embodiments only and is not intended to be limiting of the invention. As used herein, the term “and/or” includes any and all combinations of one or more of the associated listed items. As used herein, the singular forms “a,” “an,” and “the” are intended to include the plural forms as well as the singular forms, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof.

As used herein, the term “cancer or tumor” refers to the mammalian, such as a human, solid tumor or solid cancer in any site which can be detected by Magnetic Resonance Imaging (MRI).

As used herein, the term “Flow and Oxygenation Dependent (FLOOD)” and “T2-weighted MR imaging technique” refers to the clinical conventional 1.5 T or 3 T MRI scanner which can non-invasively measure tumor physiological information.

As used herein, the term “tumor microcirculation” and “tumor oxygenated perfusion” refers to the capability of fresh oxygenated blood flow through tumor region. Here, the percentage of fresh oxygenated blood flow region is used to measure parameter of tumor microcirculation.

As used herein, the term “therapeutic resistance” or “treatment resistance” or “drug resistance” refers to the drug resistance in systemic therapies. The resistance can be divided into two broad categories: cells-specific factors and pharmacological/physiological factors. The poor drug distribution/concentration is one of the factors in pharmacological/ physiological category.

As used herein, the term “blood-borne therapies” or “systemic therapy” or “systemic treatment” refers to the systemic therapies. Here, systemic therapies are drugs/agents that spread throughout the body to treat cancer cells wherever they may be. They include chemotherapy, hormonal therapy, targeted therapy, immunotherapy, gene therapy, and photodynamic therapy.

As used herein, the term “precision medicine in cancer treatment” and “precision cancer treatment” refers to a method of providing the most suitable treatment method according to the characteristics of cancer. Here, the precision cancer treatment may include all cancer therapies clinically except surgical treatment.

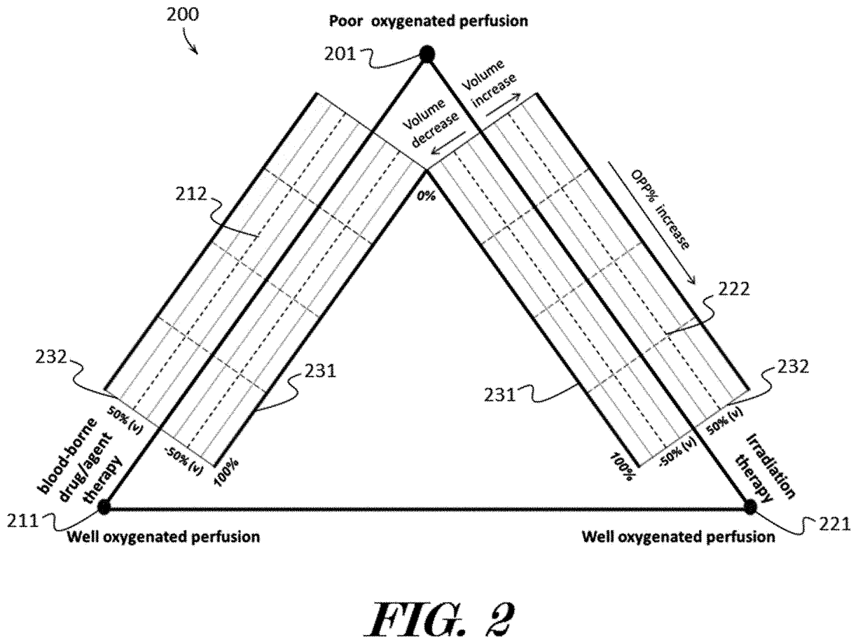

As used herein, the term “cancer treatment response diagram” or “cancer treatment response information diagram” or “cancer treatment infographic” refers to the integrating the volume of tumor change ratio Vt % and oxygenated perfusion percentage OPP % as one therapeutic response information point on the cancer treatment response information diagram as shown in FIGS. 2 and 5-9. One cancer treatment response information diagram may have several different treatment response information points during different periods of cancer treatment.

As used herein, the term “computer” refers to a machine, apparatus, or device that is capable of accepting and performing logic operations from software code. The term “application”, “software”, “software code” or “computer software” refers to any set of instructions operable to cause a computer to perform an operation. Software code may be operated on by a “rules engine” or processor. Thus, the methods and systems of the present invention may be performed by a computer or computing device having a processor based on instructions received by computer applications and software.

The term “electronic device” as used herein is a type of computer or computing device comprising circuitry and configured to generally perform functions such as recording audio, photos, and videos; displaying or reproducing audio, photos, and videos; storing, retrieving, or manipulation of electronic data; providing electrical communications and network connectivity; or any other similar function. Non-limiting examples of electronic devices include: personal computers (PCs), workstations, laptops, tablet PCs including the iPad, cell phones including iOS phones made by Apple Inc., Android OS phones, Microsoft OS phones, Blackberry phones, digital music players, or any electronic device capable of running computer software and displaying information to a user, memory cards, other memory storage devices, digital cameras, external battery packs, external charging devices, and the like. Certain types of electronic devices which are portable and easily carried by a person from one location to another may sometimes be referred to as a “portable electronic device” or “portable device”. Some non-limiting examples of portable devices include: cell phones, smartphones, tablet computers, laptop computers, and wearable computers such as Apple Watch, other smartwatches, Fitbit, other wearable fitness trackers, Google Glasses, and the like.

The term “user device” or sometimes “electronic device” or just “device” as used herein is a type of computer or computing device generally operated by a person or user of the system. In some embodiments, a user device is a smartphone or computer configured to receive and transmit data to a server or other electronic device which may be operated locally or in the cloud. Non-limiting examples of user devices include: personal computers (PCs), workstations, laptops, tablet PCs including the iPad, cell phones including iOS phones made by Apple Inc., Android OS phones, Microsoft OS phones, Blackberry phones, or generally any electronic device capable of running computer software and displaying information to a user. Certain types of user devices which are portable and easily carried by a person from one location to another may sometimes be referred to as a “mobile device” or “portable device”. Some non-limiting examples of mobile devices include: cell phones, smartphones, tablet computers, laptop computers, wearable computers such as Apple Watch, other smartwatches, Fitbit, other wearable fitness trackers, Google Glasses, and the like.

The term “computer readable medium” as used herein refers to any medium that participates in providing instructions to the processor for execution. A computer readable medium may take many forms, including but not limited to, non-volatile media, volatile media, and transmission media. Non-volatile media includes, for example, optical, magnetic disks, and magneto-optical disks, such as the hard disk or the removable media drive. Volatile media includes dynamic memory, such as the main memory. Transmission media includes coaxial cables, copper wire and fiber optics, including the wires that make up the bus. Transmission media may also take the form of acoustic or light waves, such as those generated during radio wave and infrared data communications.

As used herein the term “data network” or “network” shall mean an infrastructure capable of connecting two or more computers such as user devices either using wires or wirelessly allowing them to transmit and receive data. Non-limiting examples of data networks may include the PACS (picture archiving and communication system) internet or wireless networks or (i.e. a “wireless network”) which may include Wifi and cellular networks. These networks may use any security protocol suitable for securing patient health information and other protected information. For example, a network may include a local area network (LAN), a wide area network (WAN) (e.g., the Internet), a mobile relay network, a metropolitan area network (MAN), an ad hoc network, a telephone network (e.g., a Public Switched Telephone Network (PSTN)), a cellular network, or a voice-over-IP (VoIP) network.

As used herein, the term “database” shall generally mean a digital collection of data or information. All MRI images raw data is stored on a file system in DICOM format. The present invention uses novel methods and processes to store, link, and modify information such digital images and videos and user profile information. For the purposes of the present disclosure, a database may be stored on a remote server and accessed by a user device through the internet (i.e., the database is in the cloud) or alternatively in some embodiments the database may be stored on the user device or remote computer itself (i.e., local storage). A “data store” as used herein may contain or comprise a database (i.e. information and data from a database may be recorded into a medium on a data store).

Unless otherwise defined, all terms (including technical and scientific terms) used herein have the same meaning as commonly understood by one having ordinary skill in the art to which this invention belongs. It will be further understood that terms, such as those defined in commonly used dictionaries, should be interpreted as having a meaning that is consistent with their meaning in the context of the relevant art and the present disclosure and will not be interpreted in an idealized or overly formal sense unless expressly so defined herein.

In describing the invention, it will be understood that a number of techniques and steps are disclosed. Each of these has individual benefit and each can also be used in conjunction with one or more, or in some cases all, of the other disclosed techniques. Accordingly, for the sake of clarity, this description will refrain from repeating every possible combination of the individual steps in an unnecessary fashion. Nevertheless, the specification and claims should be read with the understanding that such combinations are entirely within the scope of the invention and the claims.

The present disclosure is to be considered as an exemplification of the invention, and is not intended to limit the invention to the specific embodiments illustrated by the figures or description below.

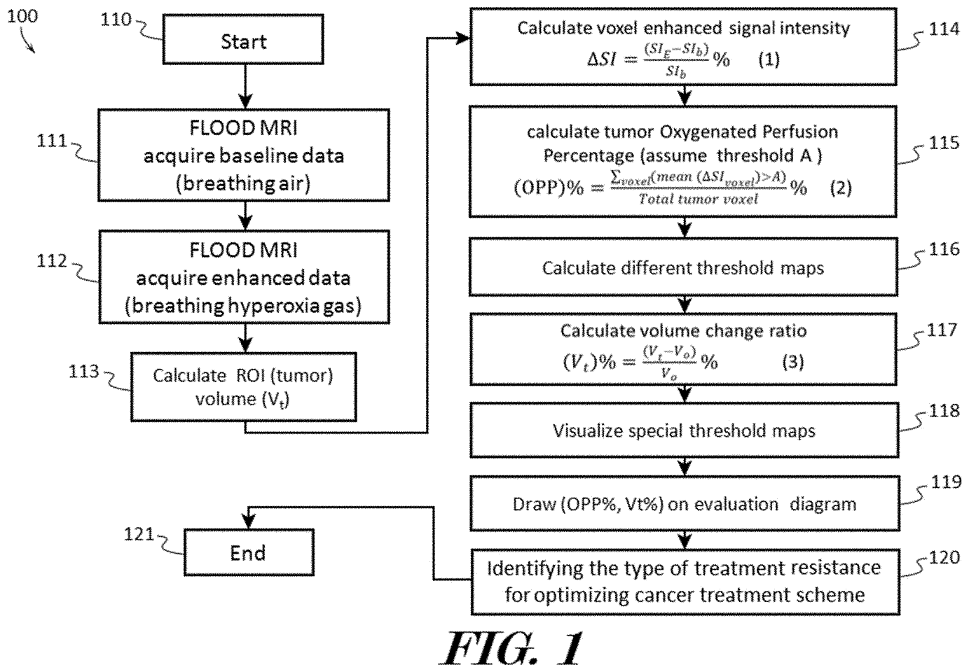

The present invention will now be described and computerized by example, algorithms, and through referencing the appended figures representing preferred and alternative embodiments. FIG. 1 illustrates a block diagram of an example of a computer implemented the identification method (“the method”) 100 according to various embodiments. FIGS. 2 and 5-9 illustrate cancer treatment response information diagrams 200 for identification of the type of drug resistance. In the following description, for purposes of explanation, numerous specific details are set forth in order to provide a thorough understanding of the present invention. It will be evident, however, to one skilled in the art that the present invention may be practiced without these specific details.

Tumor Microcirculation Characteristic and Angiogenesis Vascular System

Microcirculation is the circulation of the blood in the smallest blood vessels, present in the vasculature embedded within organ tissues. The main functions of blood in the microcirculation are the delivery of oxygen (O₂), nutrients, drug/agent and the removal of carbon dioxide (CO₂). The vascular system of tumor is totally different from that of normal tissue, which usually is abnormal vascular system and construction in the tumor. The cancer cells of one region without blood circulation grew to 1-2 mm³ in diameter and then stopped, but grew beyond 2 mm³ when placed in an area where angiogenesis was possible. Tumor angiogenesis allows for supply of oxygen, nutrients, growth factors, and tumor dissemination to distant sites. Abnormal tumor vasculature typically lacks hierarchical structure and is composed of immature differentiated and undifferentiated vessels with increased permeability. The undifferentiated vessels frequently present with either collapsed or an absent lumen. Consequently, tumor vasculature is inefficient in carrying oxygen blood flow, and inefficient tumor microcirculation resulting in inefficient drug distribution and a hypoxic tumor. The region of inefficient perfusion (microcirculation) of the tumor is associated with an inefficient drug/oxygen distribution region, which may lead to failure of systemic therapy and radiation therapy.

Special Protocol and MRI Technique For Monitoring Treatment Response

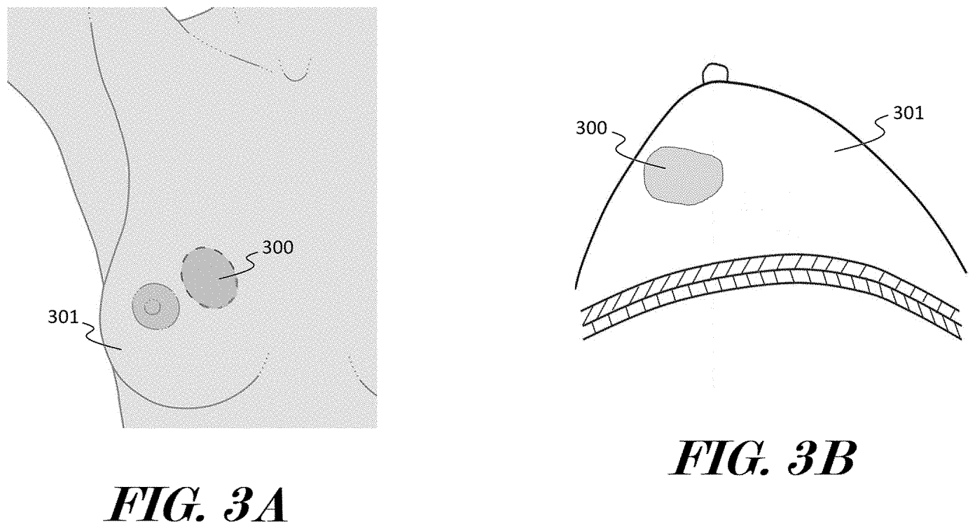

How to monitor tumor physiological response information during treatment is a challenge faced in the clinical setting. Usually, DCE (dynamic contrast enhancement) MRI technique as a diagnosis approach has been used to diagnose tumor via injecting extrinsic contrast agent Gd-DTPA in the clinic setting. The signal intensity of this imaging technique may depend on the leakage of extrinsic contrast agent from vascular system. Tumor regions with higher vascular permeability may directly result in an increase in signal intensity in the same region. In the diagnostic phase of a primary tumor, injection of an external contrast agent may be helpful in diagnosing a primary tumor. If DCE MRI technology is used to monitor the response during treatment, it will provide the wrong information because the permeability of tumor blood vessels may vary greatly under certain treatment. It has been proved by clinical studies. In preferred embodiments, the present invention may use a novel MR imaging mechanism to non-invasively detect the physiological response of a tumor during treatment. When blood flows through vessel from arteriole to venule in tissue, the HbO₂ concentration gradually decreases and deoxyhemoglobin (dHbO₂) concentration gradually increases and reaches equilibrium with tissue. In the present invention, since blood deoxyhemoglobin (dHbO₂) is paramagnetic and blood oxyhemoglobin (HbO₂) is non-paramagnetic, deoxyhemoglobin (dHbO₂) can be used as an endogenous contrast agent. Tumor physiology or interventions that affect tumor oxygenation can complement conventional clinical MRI. The Flow and Oxygenation Dependent (FLOOD) or dynamic contrast enhanced T2-weighted MR imaging technique can be performed on clinical human 1.5 T or 3 T MRI scanner system (FIG. 3). According to the imaging scheme of the present invention, when a patient inhales normal air as a reference and then change to high-oxygen gas, it may cause a high concentration of hemoglobin (HbO₂) to circulate throughout the body. The blood of high hemoglobin (HbO₂) in the tumor arteries flows to the vein and gradually decreases until it reaches the background of the oxygenation level around the tissue near the vein, and acquired a series of dynamic images by FLOOD MRI technique or dynamic contrast enhanced T2-weighted MR imaging technique throughout the process. The conversion from dHbO₂ to HbO₂ produces MR signal gain, and the magnitude of the enhancement is positively correlated with the change in dHbO₂. The advantage of a source contrast agent is that O₂ and CO₂ can be rapidly exchanged across the entire vessel wall without any vascular permeability barrier and the effects of the T2 contrast agent may decay and disappear. Comparing with tumor reference, the largest difference in dHbO₂ occurs near the tumor arterial, while the smallest difference in dHBO₂ occurs near the vein. In other words, the highest T2 contrast effect is near the tumor arterial then decay. A unique advantage of the present invention in the use of imaging techniques versus external contrast bio-imaging techniques is that only fresh oxygenated blood flow can enhance signal intensity and then decay without being affected by vascular permeability. Accurate detection of tumor physiology information during treatment is a key for analyzing cancer treatment response information. This MR imaging protocol and technique have been published.

Quantitatively Measure Tumor Microcirculation

The microcirculation is the circulation of the blood in the smallest blood vessels, the microvessels include terminal arterioles, metarterioles, capillaries, and venules. Arterioles carry oxygenated blood to the capillaries, and blood flows out of the capillaries through venules into veins. The main functions of the microcirculation are the delivery of oxygen and nutrients and the removal of carbon dioxide (CO₂). Measurement of tumor microcirculation is a valuable medical diagnostic in the clinic. As non-invasive methods, laser Doppler perfusion imaging and laser speckle contrast imaging allow non-contact measurements of microcirculation. These techniques may only allow for measurements of surface tissue. Also, the measured results cannot distinguish the fresh oxygenated blood and low HbO₂ blood inside tumor. In fact, only fresh oxygenated (high HbO₂) blood perfusion areas may be more valuable for assessing tumor microcirculation in pathophysiology. The better the fresh oxygenated blood perfusion region, the more efficient the microcirculation tumor region exchange. The ability to quantitatively assess tumor oxygenated perfusion (tumor microcirculation) is a novel aspect of the present invention.

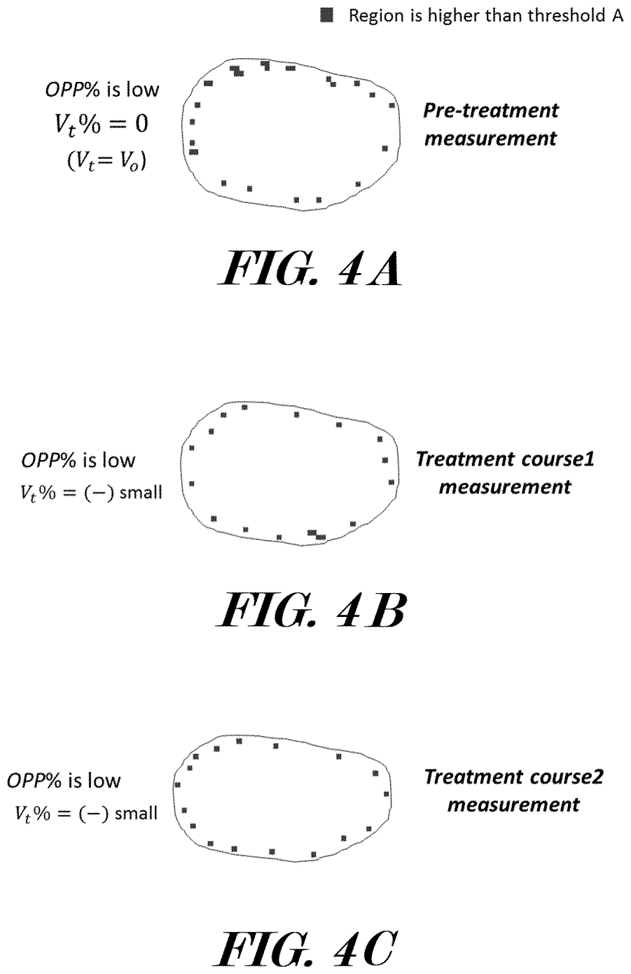

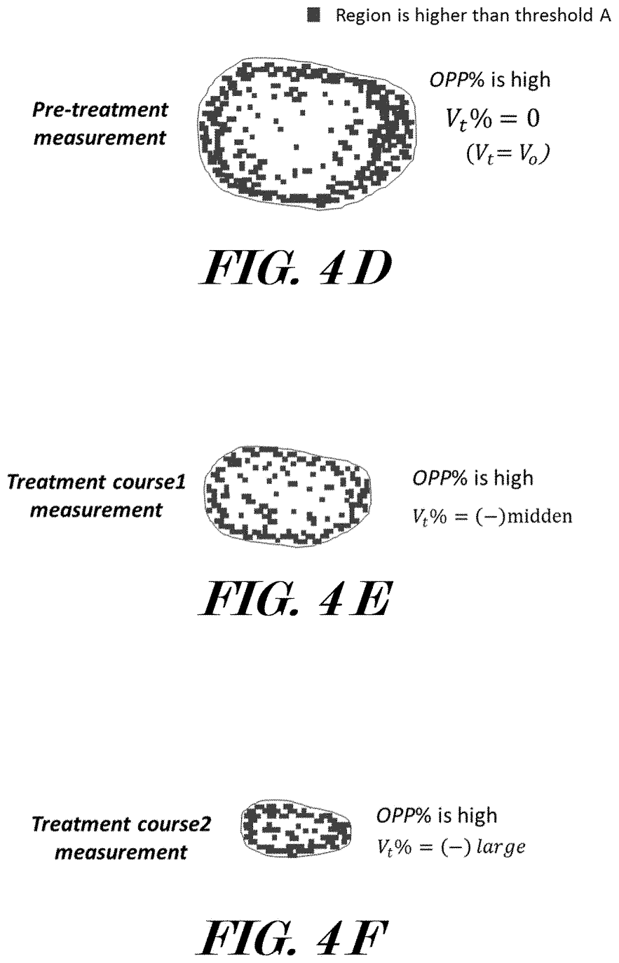

Based on MR imaging protocols and techniques, data processing may include automatically contouring tumor ROI region, calculating enhanced signal intensity of each voxel of tumor region. In some embodiments, the relative signal intensity (ΔSI) of tumor on a voxel-by-voxel basis may be calculated using the equation (from common knowledge):

$$Δ SI = \frac{( {SI}_{E} - {SI}_{b} )}{{SI}_{b}} \% \qquad ( 1 )$$

Where, SI_{E} refers to the average of enhanced signal intensity during breathing hyperoxia gas and SI_{b} is defined as the average of the baseline images in same voxel breathing air. The relative signal intensity (ΔSI) tumor area may represent the region with a high T2 contrast enhancing effect. In order to evaluate and analyze the fresh oxygenated perfusion region, a threshold A may be selected and the high contrast enhanced signal (ΔSI) may be classified voxel-by-voxel using threshold A. The accumulation of all voxels with a relative signal strength (ΔSI) above the threshold A may be used to calculate their volume percentage, so called oxygenated perfusion percentage (OPP %). The OPP % can quantitatively represent the parameters of the degree of microcirculation effectiveness of the tumor. In preferred embodiments, oxygenated perfusion percentage data OPP % is to use threshold technique in processing dynamic contrast enhancement T2 weighted MRI data for quantitatively measuring patient tumor microcirculation during the course of treatment or before treatment. High (OPP %) means that more regions of the tumor flowing through fresh oxygenated blood, which represents better drug/agent/oxygen delivery and distribution. The oxygenated perfusion percentage (OPP %) of tumor can be quantified by following equation:

$$( OPP ) \% = \frac{{\sum }_{voxel} ( mean ( Δ {SI}_{voxel} ) > A )}{Total tumor voxel} \% \qquad ( 2 )$$

Where, the threshold A is selected as a percentage based on the MR imaging pulse sequence, TR/TE time, magnet strength of clinical scanner, sensitivity of coil, cancer site, and etc. For example, it can be assumed a standard threshold 10% for 1.5 T and 15% for 3 T MRI scanner. The lower OPP % may represent inefficient drug/agents' distribution which may be associated with ineffective treatment outcomes (FIGS. 4A-4C). Conversely, the higher OPP % of tumor may be, the better drug/agent's distribution and better microcirculation which may be associated with effective treatment outcomes (FIGS. 4D-4F).

For analysis of tumor microcirculation distribution, a multiplate threshold set may be used to generate the map with different threshold which is the OPP % pseudo color image for better visualization. The tumor pseudo color map may provide another way for visualizing tumor microcirculation distribution map.

Herein, although tumor microcirculation may be quantified by the OPP % parameter which is the relative value of each patient's inhaled hyperoxia gas, the OPP % cannot be used to measure the value of plasma drug concentration in tumor region. For example, the low OPP % of the tumor is related to the relatively small amount of fresh oxygenated blood flowing through and the relatively low drug/oxygen delivery capacity. It cannot be used to quantitatively measure the plasma drug concentration and the absolute value of pO₂ in local region of the tumor. For radiotherapy, a lower OPP % case may only indicate the potential for hypoxia, which may lead to treatment barrier in radiation therapy. The advantage of using the relative value of OPP % is that the tumor microcirculation of all different tumors can be quantified and unified into a set of criteria for further evaluation.

Two Different Parameters May Serve as One Treatment Response Information Point

Tumor volume responses to effective treatment can often be clinically delayed for days or weeks. As an important parameter, changes in tumor volume have been widely used to assess previous effects during treatment. Traditional X-ray, ultrasound, and CT diagnostic techniques are used to monitor changes in tumor volume. Although tumor volume delays in response to treatment are common behaviors, tumor volume information remains valuable in assessing previous treatments. Here, the relative tumor volume change rate Vt % is used to measure the change in tumor volume based on the T2-weighted MRI images.

The tumor volume pre-treatment V₀ is defined as reference of volume. Each measurement of tumor volume during treatment can be compared with reference and calculated:

$$( {V}_{t} ) \% = \frac{( {V}_{t} - {V}_{o} )}{{V}_{o}} \% \qquad ( 3 )$$

Where, V₀ is the tumor reference volume at pre-treatment; Vt is the volume of tumor response to treatment during measurement. The first measurement of the pretreatment, Vt %=0. When the tumor shrinks, Vt % shows a negative value. If the tumor completely responds to treatment and disappears, Vt %=−100%. If the tumor increases during treatment, Vt % shows a positive value.

In order to better monitor cancer treatment, changes in tumor volume are not sufficient to timely reflect the response of the tumor to treatment. There are many uncertainties in treatment. For example, tumor atrophy may lead to changes in the internal hemodynamics and microcirculation patterns of the tumor, which may directly lead to drug resistance in further treatment. To monitor the dynamic change of tumor microcirculation during treatment course shows a significant meaning in cancer treatment. In fact, clinicians need to understand information about two different categories of treatment response, the previous treatment response Vt %, and the likely future response information OPP % (prognosis). In the present invention, these two parameters have been used as a therapeutic response point on a particular coordinate system. More importantly, clinicians are given the opportunity to assess resistance in order to adjust treatment options and achieve accurate evidence-based cancer treatment. Another advantage of the two-dimensional response data style is the demonstration of the ability to identify the type of resistance factor during treatment. This may be the first time that a tumor's previous response and possible future response will be introduced as a therapeutic response information point for analyzing treatment effects in clinical routine.

Uniform Criteria for Reviewing Therapeutic Information

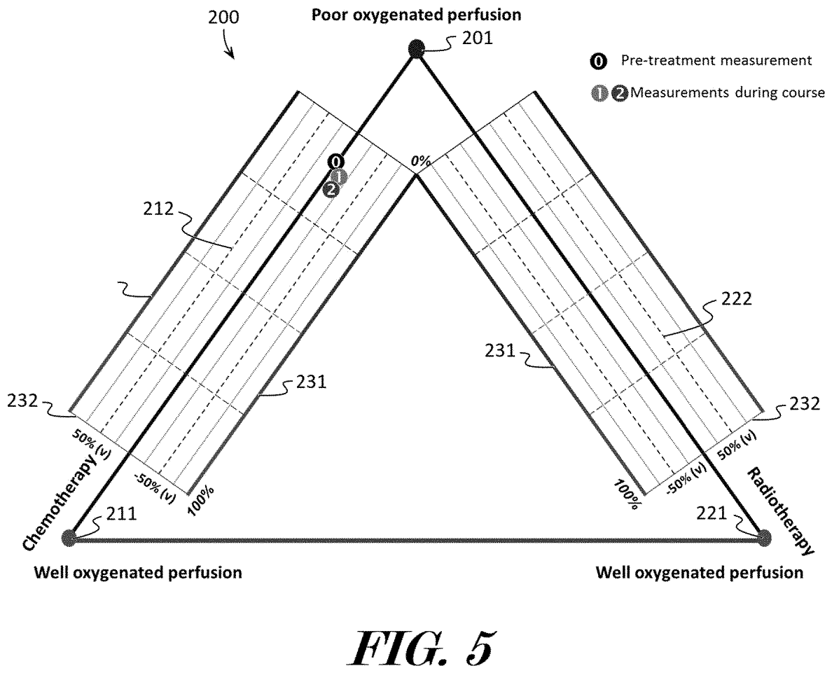

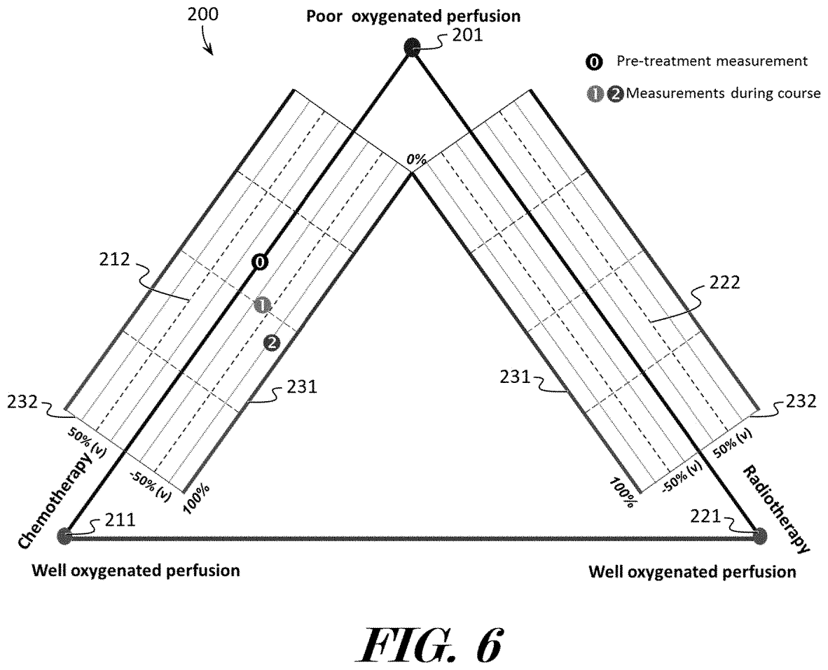

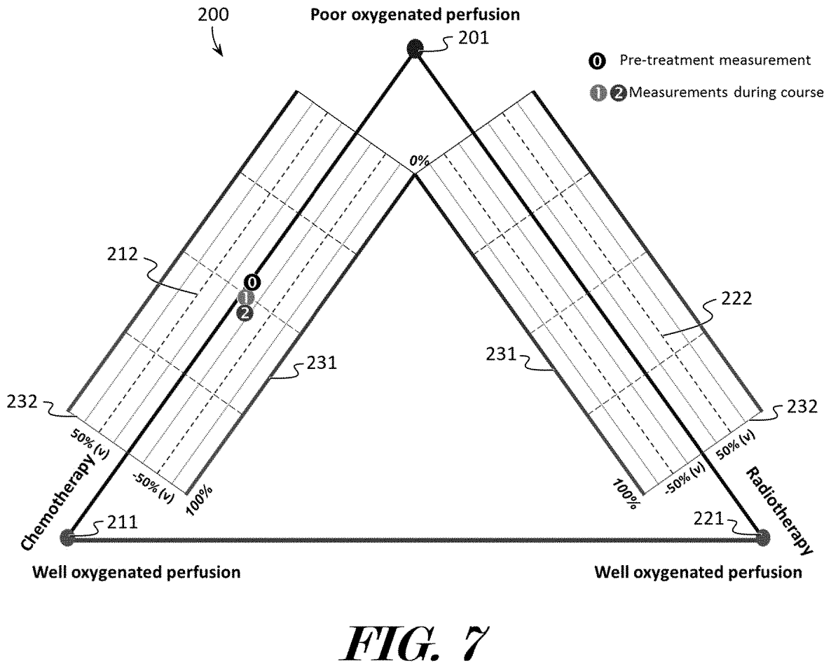

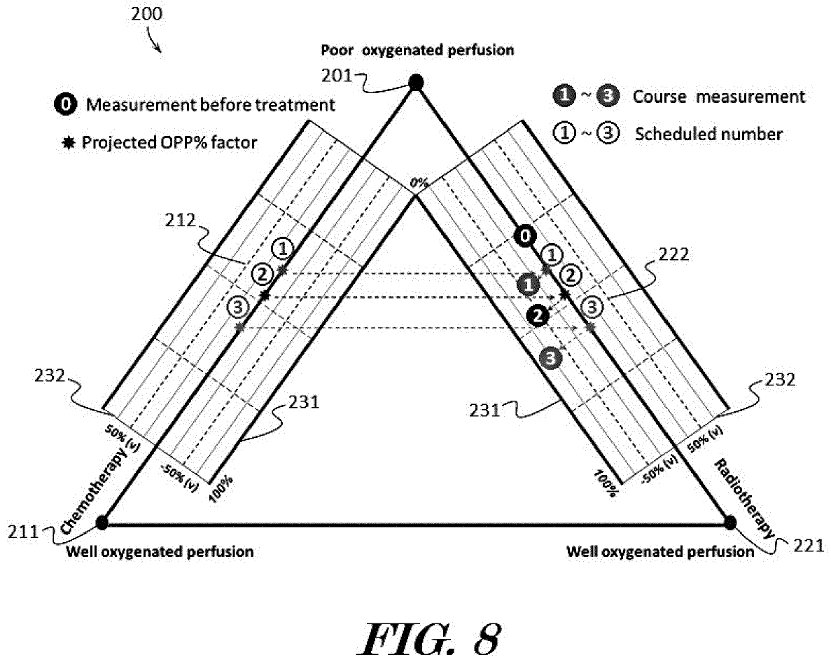

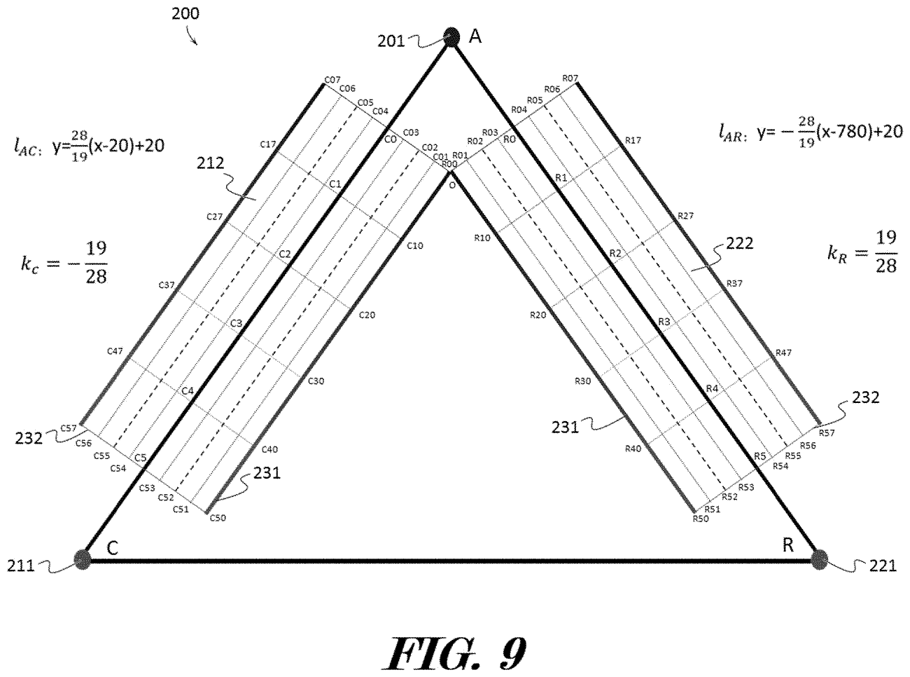

Cancer is a complex disease. A single treatment therapy is rarely able to cure the disease clinically. It may require multiple therapies to treat together. It may require a common platform to aggregate information about each treatment response for tracking, evaluation, and sharing with clinicians who have different therapeutic backgrounds. In addition to surgery, the results of systemic therapies and local radiotherapy are associated with local microcirculation of the tumor. In the present invention, a special infographic has been devised for displaying tumor treatment response information (FIG. 2). The cancer treatment response information diagram (“the diagram”) 200 comprises two symmetric coordinate systems. Left side represents the systemic therapy response information, right side the local treatment (radiotherapy) response information. Unified standards for infographics have been used to monitor, evaluate, and track treatment responses (FIGS. 5-8). Two breast cancer cases (FIGS. 4A-4C and FIGS. 4D-4F) and their response to chemotherapy are shown in cancer treatment response information diagrams 200 of FIG. 5 and FIG. 6. FIGS. 3A and 3B show examples of a breast cancer tumor 300 and breast 301. In FIGS. 4A-4F, gray area indicates that the area is greater than the threshold, and generating a gray map uses only a single threshold. FIGS. 4A-4C shows an example of an ineffective treatment situation where OPP % is low and the tumor volume reduces small during treatment. FIGS. 4D-4F shows an example of an effective treatment situation where OPP % is high and tumor volume decreases large. According to the change of Vt % following treatment, it is easy to assess ineffective chemotherapy (FIG. 5) and effective treatment (FIG. 6). Additionally, FIG. 6 may demonstrate a good or positive trend because of high OPP %. In clinic, the high OPP % (better tumor microcirculation) case may or may not contribute to the better treatment effects. As shown FIG. 7, a situation that well tumor microcirculation doesn't respond to chemotherapy or targeted therapy and shows an ineffective treatment is demonstrated. Based on the two symmetric coordinate system of infographic, clinicians may review, and analyze the cancer treatment case with chemotherapy combining radiotherapy (FIG. 8). In the cancer treatment response information diagram 200, the tumor's treatment response information point of all measurements during treatment may be dynamically displayed on one cancer treatment response information diagram 200 for evaluating cancer treatment strategy in clinical setting. The detailed design specification of the cancer treatment response information diagram 200 is shown in FIG. 9.

Identifying the Type Of Drug Resistance

If cancer treatment is not effective, the clinician must know the cause of the treatment disorder and adjust the treatment plan in time. Medical research has shown that the resistance of systemic therapy can be divided into two categories: cell-specific factors and pharmacological/physiological factors. The low drug distribution/concentration is one of the drug resistances of pharmacological/physiological factors. Two different types of resistance may require different clinical strategies to overcome their barriers. At the same time, clinical studies have shown that most human solid tumors exhibit inefficient microcirculation. In other words, most systemic treatment cases may show treatment failure due to low drug distribution/concentration. Precision medicine for cancer is an approach to deliver the most appropriate treatments based on the characteristics of each cancer. How to identify drug resistance is essential for adopting the right treatment strategy to overcome treatment barriers and minimize ineffective treatment.

Unfortunately, current clinicians may not have this important information in developing a treatment plan during treatment, which may result in clinically ineffective treatment or even ineffective overtreatment.

In the present invention, the type of therapeutic resistance can be identified in time by analyzing the cancer treatment response information diagram 200. The identification process may be as follows: for example, if the tumor microcirculation parameter (OPP %) is less than 5%, treatment resistance can be considered as a high probability event due to low drug distribution/concentration resistance (FIG. 5). Actually, the drug resistance from low distribution/concentration is able to be identified prior to systemic treatment (FIG. 5). During chemotherapy, these two measurements still showed low OPP % and Vt % is less than 3%, which confirmed that the tumor was low drug distribution resistance (FIG. 5). It will be time for the clinician to decide to stop continuous chemotherapy without having to wait until all chemotherapy is completed.

When the tumor microcirculation parameter (OPP %) is always more than 20% and the tumor volume change (Vt %) is less than 3%, for example, it can be considered that the drug resistance is likely to be caused by cell-specific factors (FIG. 7). To determine the resistance of a cell-specific factor, a few measurements may be required during treatment. As shown in FIG. 7, two treatment response measurements may be required to confirm the type of resistance. It will be time for the clinician to decide to replace therapeutic agent/drug. Under this circumstance, clinicians may still have the opportunity to modify treatment strategies to improve cancer treatment.

Herein, repeating the results of two measurements may have the consistency in identifying the type of drug resistance. It is sufficient for final identification and reduces ineffective treatment (FIGS. 5 and 7). Criteria for identifying drug resistance, such as threshold values for determining OPP % and Vt % of drug resistance types, may depend on analysis of large clinical databases. The present invention may provide novel technical approaches and methods for identifying types of therapeutic resistance. The present invention can achieve a timely clinical identification of the type of drug resistance during cancer treatment, improving current cancer treatment techniques and methods.

Computerizing the Invention for Clinical Application

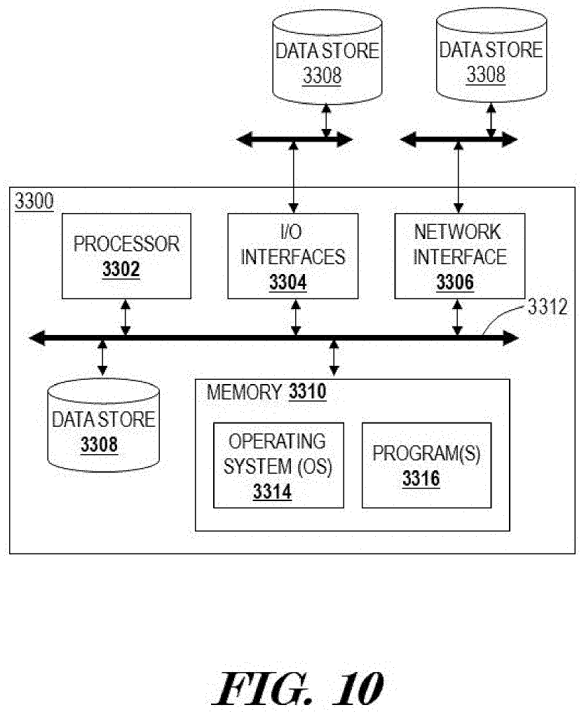

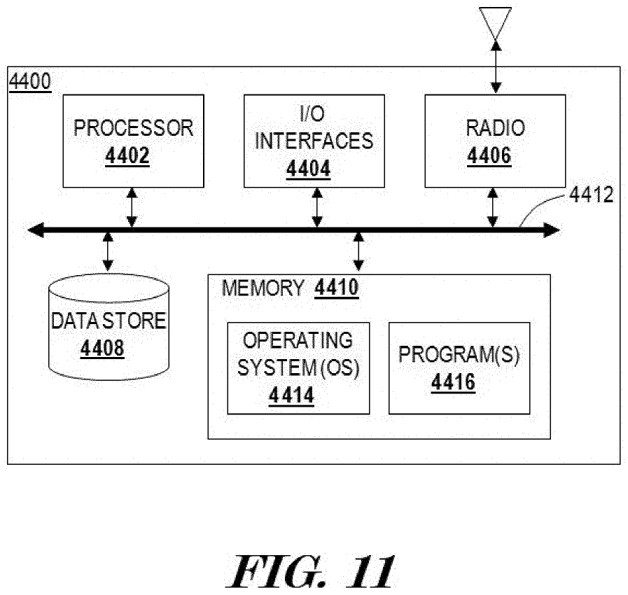

The present invention will now be described and computerized by example, algorithms, and through referencing the appended figures representing preferred and alternative embodiments. FIG. 1 illustrates a block diagram of an example of a computer implemented the identification method (“the method”) 100 according to various embodiments. In some embodiments, one or more steps 110-120 may be performed on an electronic device 4400 (FIG. 11) and/or on a server 3300 (FIG. 10). The method 100 may be used to create a cancer treatment response information diagram 200 (FIGS. 5-8) for treatments including, but not limited to Blood-borne systemic therapies, such as Chemotherapy, Molecularly Targeted therapy, Immunotherapy; Gene therapy, and Photodynamic therapy, Irradiation therapies, such as Radiotherapy, and Combination therapies, such as chemotherapy-radiotherapy, immunotherapy-radiotherapy, molecularly targeted therapy-radiotherapy, radiosensitizer-radiotherapy, other Blood-borne systemic therapies-irradiation therapies for a particular patient 501. In some embodiments, one or more steps 110-121 may be performed before, during, or after a cancer therapy treatment. In further embodiments, one or more steps 110-121 may be performed during, before, or after a cancer therapy treatment scheme. In some embodiments, the method 100 may be used for the treatment of human solid tumors, although in further embodiments, the method 100 may be used for the treatment of solid tumors in any mammal or other organism.

In some embodiments, the method 100 may start 110 and the tumor oxygenated perfusion may be detected by using a Flow and Oxygenation Dependent (FLOOD) contrast MRI (dynamic contrast enhanced T2 weighted MR imaging) technique, which is sensitive to which is sensitive to both vascular oxygenation and flow. In further embodiments, the tumor oxygenated perfusion may be detected having the patient breathe air to acquire baseline data in step 111. Next, after inhalation of hyperoxia gas to generate endogenous contrast agent and higher than baseline HbO₂ blood circulating in body, the enhanced data may be acquired in step 112. When higher HbO₂ blood flow through tumor region comparing with difference of HbO₂ between baseline breathing air and hyperoxia gas in same region, the dynamic T2-weighted MRI technique can detect an enhanced MRI signal intensity which is positively related to difference range of HbO₂ in same region. In further embodiments, the pre-treatment MRI measurement may be taken as control and compared with following measurements during the course of treatment. The tumor volume (V₀) of pre-treatment measurement may serve as a control for calculating volume change ratio during evaluation of the course of treatment. The step 111 and step 112 are from published papers (common knowledge).

In further embodiments, step 111 and/or step 112 may be performed by an Input/Output (I/O) Interface 4404 (FIG. 11), 3304 (FIG. 10), of a server 3300 and/or an electronic device 4400. The data acquired in steps 111 and 112 may be stored in a data store 4408 (FIG. 11), 3308 (FIG. 10), and be accessible to a processor 4402 (FIG. 11), 3302 (FIG. 10). The processor 4402, 3302, may then calculate the region-of-interest (ROI) volume (Vt) of the tumor, which may be performed by volume contour tracing/region-of-interest (ROI) analysis 3D tumor volumetry methods in step 113 which is based on intensity threshold of the T2-weighted Mill images. The tumor regions generally show relatively high signal intensity in T2-weighted Mill images comparing with around normal tissue. The tumor ROI region define and alone gave unacceptable overlap of intensity distributions for tumor and normal tissue. In some cases, it may need to do original data processing for motion correction before analyzing data. The step 113 is from common knowledge.

Next, in step 114, the processor 4402 (FIG. 11), 3302 (FIG. 10) may calculate voxel's enhanced signal intensity (ΔSI). In some embodiments, data analysis may be performed on a voxel-by-voxel basis.

The relative signal intensity (ΔSI) of each tumor voxel may be analyzed using the equation:

$$Δ SI = \frac{( {SI}_{E} - {SI}_{b} )}{{SI}_{b}} \% \qquad ( 1 )$$

Where, SI_{E} refers to the enhanced signal intensity in the voxel and SI_{b} is defined as the average of the baseline images in same voxel. The mean signal intensity-time curve of tumor is used to evaluate quality of measurement. The smooth processing is used to eliminate unstable points due to patient motion. The step 114 is from common knowledge. In step 115, tumor oxygenated perfusion percentage (OPP) may be calculated by the processor 4402 (FIG. 11), 3302 (FIG. 10). The threshold A may be selected as classify high and low contrast enhanced signal (ΔSI) in voxel basis in order to assess whole tumor oxygenated perfusion status. The voxels of the relative signal intensity (ΔSI) being higher than threshold A is counted as high oxygenated perfusion voxel. The percentage of the higher oxygenated perfusion voxel is counted and defined as parameter for evaluating tumor oxygenated perfusion. The higher oxygenated perfusion percentage represents tumor with more oxygenated perfusion inside tumor and better drug/agent/oxygen delivery and distribution. The oxygenated perfusion percentage factor of tumor can be quantified by following equation:

$$( OPP ) \% = \frac{{\sum }_{voxel} ( mean ( Δ {SI}_{voxel} ) > A )}{Total tumor voxel} \% \qquad ( 2 )$$

Where, the threshold A is selected as a percentage based on the MR imaging pulse sequence, TR/TE time, magnet strength of clinical scanner, sensitivity of coil, cancer site, and etc. For example, it can be assumed a standard threshold 10% for 1.5 T and 15% for 3 T MRI scanner. The OPP % factor represents the how many percent tumor regions with oxygenated perfusion above threshold level A, which is an important prognostic factor for next systemic therapy and can be dynamic changed with treatment course. The higher OPP % represents tumor with the better oxygenated perfusion. Conversely, the lower OPP % represents the lower oxygenated perfusion in tumor region, thereby clarifying the prognostic value of tumor oxygenated blood perfusion.

Next, in step 116, the different threshold set can be processed and different threshold maps may be calculated by processor 4402 (FIG. 11), 3302 (FIG. 10) such as a reconstruction OPP % pseudo color image for better visualization. Several threshold values (such as 0%, 5%, 10%, 20%, and 30%) can be used to classify each voxel and respectively pseudo color value. For example, assign different pseudo color values (1, 50, 100, 150, 200, 250) to voxel's relative signal intensity (ΔSI) respectively (<0%, 0%˜5%, 5%˜10%, 10%˜20%, 20%˜30%, >30%) which respectively correspond to a color table (dark blue, blue, light blue, brown, purple, red). Each voxel of tumor only has one pseudo color value. The tumor pseudo color map data set is completed for display on a display input/output device 3304, 4404. Meanwhile the different threshold values may be used to process in step 115 for calculating different threshold OPP % histogram map for analyzing previous treatment response.

In step 117 the tumor volume change ratio (Vt %) may be calculated by the processor 4402 (FIG. 11), 3302 (FIG. 10). The total tumor volume before treatment is defined as original volume V₀. Each measurement of tumor volume during treatment can be calculated by accounting tumor region Vt.

$$( {V}_{t} ) \% = \frac{( {V}_{t} - {V}_{o} )}{{V}_{o}} \% \qquad ( 3 )$$

Where, V₀ is the tumor original volume at pre-treatment; Vt is the volume of tumor response to treatment. The first measurement of the pretreatment, Vt %=0. When the tumor shrinks, Vt % shows a negative value. If the tumor completely responds to treatment and disappears, Vt %=−100%. If the tumor increases during treatment, Vt % shows a positive value. The Vt % parameter directly correlates to cancer response to previous treatment. The step 117 is from common knowledge.

In step 118, special threshold maps may be created by the processor 4402 (FIG. 11), 3302 (FIG. 10) to visualize the data such as using reconstruction OPP % to form pseudo color image of the data in step 116. The three-dimensional pseudo color map data set can be used to visualize tumor different oxygenated perfusion distribution and therapeutic response. 2D pseudo images may displayed as slice by slice on an I/O Interface 4404 (FIG. 11), 3304 (FIG. 10), printer or display screen of a server 3300 and/or an electronic device 4400. The image may be displayed either all pseudo color or only interested pseudo colors image for visualization. For example, by selecting brown, purple, and red color, it may display tumor high oxygenated perfusion area which may be used to evaluate the tumor prognostic information. The dark blue and blue regions correlate to regions of low/non oxygenated perfusion. By selecting dark blue, blue, and light blue colors it may display the tumor low/non oxygenated perfusion image which may be used to monitor the change this part during the course of treatment. As low/non oxygenated perfusion regions of tumor displaying dark blue, blue, and light blue color region of images, they may be fussed to radiation treatment plan for functional image guided irradiation therapy. Next in step 119, the OPP % and Vt % may be drawn on a cancer treatment response information diagram 200 (FIGS. 5-8) which may be displayed on an I/O Interface 4404, 3304, printer or display screen of a server 3300 and/or an electronic device 4400. In some embodiments, a reconstruction tumor oxygenated perfusion percentage OPP % pseudo color image may be displayed or during the course of the cancer treatment on a display input/output device 4404, 3304. In further embodiments, the oxygenated perfusion percentage data OPP % and volume change ratio Vt % obtained before a cancer treatment course may be plotted and the oxygenated perfusion percentage data OPP % and volume change ratio Vt % obtained during the cancer treatment course may be plotted on the cancer treatment response information diagram 200. In still further embodiments, the oxygenated perfusion percentage data OPP % and volume change ratio Vt % data obtained during a first cancer therapy for a particular patient 501 may be plotted on the first change in tumor volume coordinate graph 212 extending from the poor oxygenated perfusion apex 201 and the first well oxygenated perfusion apex 211 of the cancer treatment response information diagram 200, and wherein the oxygenated perfusion percentage data OPP % and volume change ratio Vt % data obtained during a second cancer therapy for the particular patient 501 may be plotted on the second change in tumor volume coordinate graph 222 extending from the poor oxygenated perfusion apex 201 and the second well oxygenated perfusion apex 221 of the cancer treatment response information diagram 200.

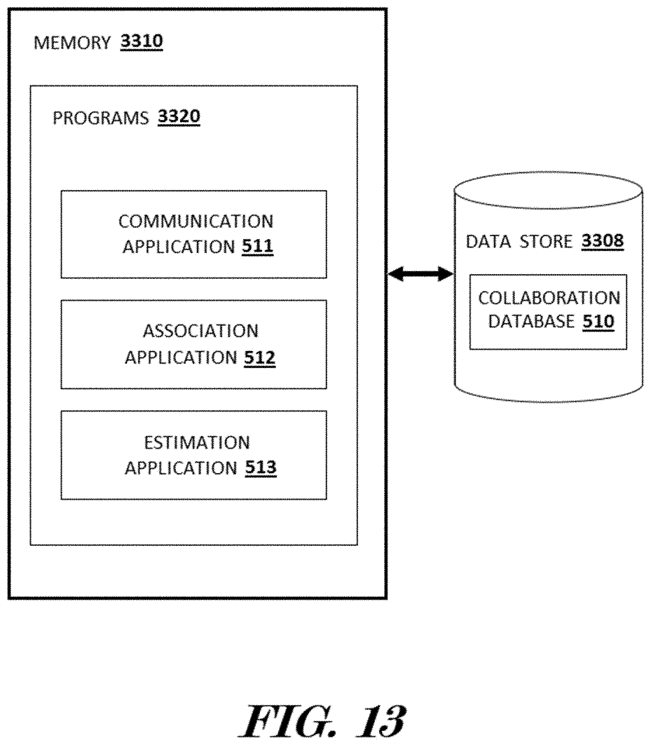

Next in step 120, the type of treatment resistance may be identified by an estimation application 513 (FIG. 13) based on the pooled cancer therapy data of one or more other patients which may be stored in a patients' database 510 (FIG. 13). If tumor microcirculation parameter (OPP %) is lower than 5% and the change of tumor volume (Vt %) is less than 3%, for example, it can be identified the treatment resistance caused by low drug distribution in systemic therapies (FIG. 4A, FIG. 5). The resistance of low drug distribution can be detected and identified before systemic treatments. If tumor microcirculation parameter (OPP %) is always more than 20% and the change of tumor volume (Vt %) are still lower than 3% in two consecutive measurements, for example, the drug resistance can be identified as the type of cells-specific factors (FIG. 7). Based on the analysis of patients' database, the standard of identification can be established for classifying the resistance of low drug distribution factor or cells-specific factors. In further embodiments, the oxygenated perfusion percentage data OPP % and volume change ratio Vt % data for a particular patient 501 may be compared to a database, such as a collaboration database 510, containing a pool of cancer therapy data, oxygenated perfusion percentage data OPP %, and volume change ratio Vt % for one or more other patients 501 to provide an identification of the treatment resistance analysis for a cancer systemic therapy to the particular patient After step 120, the method 100 may end 121.

FIG. 9 provides an example construction of a cancer treatment response information diagram 200 according to various embodiments described herein. It should be understood that a cancer treatment response information diagram 200 may be drawn or composed in any shape, but to further understanding of the invention, some example equations are provided which may be used to construct all or portions of a cancer treatment response information diagram 200. In this example, the diagram 200 may be constructed with an area of 800 by 600 pixels, although other sizes and scales may be used, with the coordinates of A being (400,580), C being (20,20), R being (780,20), R00 being (400,437), C00 being (400,437), O being (400,437).

In some embodiments, the 201 to 211 side (side AC) of the diagram 200 may be drawn according to the following equation:

$${l}_{A C} \text{:} y = \frac{28}{19} ( x - 20 ) + 20$$

In some embodiments, the slope of the 201 to 211 side (side AC) of the diagram 200 may follow the equation:

$${k}_{c} = - \frac{19}{28}$$

In some embodiments, the 201 to 221 side (side AR) of the diagram 200 may be drawn according to the following equation:

$${l}_{AR} \text{:} y = - \frac{28}{19} ( x - 780 ) + 20$$

In some embodiments, the slope of the 201 to 221 side (side AR) of the diagram 200 may follow the equation:

$${k}_{R} = \frac{19}{28}$$

FIG. 2 illustrates an example of a novel cancer treatment response information diagram (“the diagram”) 200 of infographic according to various embodiments described herein. In some embodiments, the diagram 200 may comprise two independent symmetrical coordination systems as a triangle structure comprising three apexes which may be oriented to different cancer therapy modalities. The poor oxygenated perfusion apex 201, optionally oriented at the top of the triangle, may indicate cancer tumors with poor oxygenated perfusion, a first therapy-well oxygenated perfusion apex 211, and a second therapy-well oxygenated perfusion apex 221, optionally oriented at the bottoms of the triangle, may indicate cancer tumors with well oxygenated perfusion. In this non-limiting example, the first therapy-well oxygenated perfusion apex 211 is used to graph blood-borne therapy data, and the second therapy-well oxygenated perfusion apex 221 is used to graph irradiation therapy data. In other embodiments, data of any therapy may be graphed on any desired apex or side of the diagram 200. Additionally, a change in tumor volume coordinate graph 212, 222, may extend from both of the two sides, such as the 201 to 211 side and the 201 to 221 side of the diagram 200. In this manner the 201 to 211 side and the 201 to 221 side of the diagram 200 may be used as a coordinate graphing system which each side functioning as a coordinate graphing system for a type of cancer therapy or treatment. For example, the first change in tumor volume coordinate graph 212 of the 201 to 211 side may function as a graphing system for a blood-borne drug/agent therapy and the second change in tumor volume coordinate graph 222 of the 201 to 221 side may function as a graphing system for an irradiation therapy. In further embodiments, the diagram 200 may have any number of sides and each side may represent any therapy.

Preferably, each change in tumor volume coordinate graph 212, 222, may comprise an oxygenated perfusion percentage (OPP %) x-axis 231 which may be used to graph oxygenated perfusion percentage (OPP %) data and each change in tumor volume coordinate graph 212, 222, may also comprise a tumor volume change ratio (Vt %) y-axis 232 which may be used to graph tumor volume change ratio (Vt %) data. In this example, negative values on the tumor volume change ratio (Vt %) y-axes 232 may be plotted inside the triangular shaped diagram 200, while positive values on the tumor volume change ratio (Vt %) y-axes 232 may be plotted outside the triangular shaped diagram 200. Also in this example, smaller values on the oxygenated perfusion percentage (OPP %) x-axes 231 may be plotted closer to the poor oxygenated perfusion apex 201 of the triangular shaped diagram 200, while greater values on the oxygenated perfusion percentage (OPP %) x-axes 231 may be plotted closer to the first 211 and second 221 therapy-well oxygenated perfusion apexes of the triangular shaped diagram 200. In alternative embodiments, the orientations and graduations of the oxygenated perfusion percentage (OPP %) x-axes 231 and/or tumor volume change ratio (Vt %) y-axes 232 may be switched, inverted, or otherwise rearranged.

Each measurement, such as those recorded in steps 115 and 117 of the method 100 (FIG. 1) may result with two values (the cancer oxygenated perfusion percentage (OPP %)) and the volume change ratio (Vt %) which may be expressed as one solid point in the coordinate system. A long axis, such as the 201 to 211 side and the 201 to 221 side, of the coordination system between 0%-100% represents tumor parameter OPP %, which the higher OPP % value correlates higher oxygenated blood perfusion and better drug/agent/oxygen delivery in tumor region and relative high dose distribution and oxygenation level around vessel. The short axis extending from the two sides of coordination system between −100%˜100% represents the therapeutic response in volume domain, where −100% means a clinical complete response and volume change ratio between −100%˜−30% means tumor shrinkage and shows clinical partial response as shown in FIGS. 5-8, change between −30%˜0% means clinical stable and positive percentage means an increase of tumor volume during treatment. If a cancer has therapeutic complete response, the OPP % of the treatment response information point is marked using previous OPP % value and Vt % of the treatment response information point is −100% . The OPP factor on long axis (201 to 211 side and the 201 to 221 side) represents cancer prognostic information correlating to next outcomes; the volume ratio on short axis (extending from both short axes) represents the cancer response to previous treatment.

The two separated coordination systems may be used to evaluate two different treatment modalities. For example, the left side (201 to 211 side) of a triangular diagram 200 may be assigned to evaluate treatment modalities or treatment schemes which are mostly depending on blood-borne system therapeutic molecules, particles, and cells therapies (such as chemotherapy, immunotherapy, gene therapy, photodynamic therapy, and developing molecularly targeted therapy, etc.), while the right side (201 to 221 side) of the triangular diagram 200 may be assigned to evaluate local irradiation therapy modalities (such as, hyperthermia therapy, radiation therapy, etc.). In some embodiments, the left side (201 to 211 side) of a triangular diagram 200 may be assigned to evaluate a first cancer therapy treatment modality or treatment scheme and the right side (201 to 221 side) of a triangular diagram 200 may be assigned to evaluate a second cancer therapy treatment modality or treatment scheme. A cancer therapy treatment modality or treatment scheme may include, but is not limited to, chemotherapy, molecular targeted therapy, immunotherapy, gene therapy, photodynamic therapy, radiation therapy, hyperthermia therapy, chemotherapy-radiotherapy combinations, molecular targeted therapy-radiotherapy combinations, immunotherapy-radiotherapy combinations, gene therapy-radiotherapy combinations, photodynamic therapy-radiotherapy combination, radiosensitizer-radiotherapy combination.

Turning now to FIGS. 5-8, oxygenated perfusion percentage data OPP % and volume change ratio Vt % from one, two, three, four, five, six, seven, eight, or more treatment course time points, such as during a cancer therapy treatment course or treatment scheme may be plotted on a cancer treatment response information diagram 200

The cancer treatment response information diagrams 200 of two cases (FIGS. 4A-4C, 4D-4F) in chemotherapy are shown in FIGS. 5 and 6. The lower oxygenated perfusion percentage (OPP %) demonstrates lower ability in drug/agent delivery, and lower dose concentration distribution in tumor region and following ineffective treatments (FIG. 5). The higher oxygenated perfusion percentage case correlates more effective drug/agent delivery and higher dose/agent concentration distribution and better outcomes (FIG. 6).

FIG. 7 illustrates an example of a cancer treatment response diagram 200 which describes an ineffective chemotherapy or targeted therapy according to various embodiments described herein. As shown in FIG. 7, although high OPP % values of the two consecutive measurements were taken during treatment, the volume ratio of tumor Vt % only changed a small amount. In this situation, drug resistance may be identified as cell-specific factors. With development of targeted therapy, the mutation of cancer cells may often cause the failure of treatment, which has been reported in professional publications. This case may demonstrate the new method of the present invention to identify the drug resistance of cells-specific factors in clinical routine. It will provide patients and clinicians more time and opportunities to adjust treatment strategy for precision cancer treatment. FIG. 8 shows an example of a cancer treatment response information diagram 200 which describes an effective chemo-radiotherapy combination cancer treatment according to various embodiments described herein. The OPP % value being projected in both asymmetric both coordination systems represent the ongoing chemotherapy and radiotherapy; the tumor volume parameter is marked at right side for evaluating the combination therapy results.

Combination cancer therapy is an effective treatment modality that has been widely used in clinical routine. This systemic treatment plus local treatment modality (such as, chemotherapy-radiotherapy and immune-radiotherapy etc.) can use both coordination systems on a triangular diagram 200 for tracking and evaluation. For example, the tumor OPP % information can be marked on the long axis of left side (the 201 to 211 side) of the coordination system, which means an ongoing chemotherapy or immunotherapy. The symmetrical position on the long axis of right side (the 201 to 221 side) of the coordination system also is projected the same marker of OPP %. Combining tumor volume information Vt %, one solid point is determined and marked on right coordination system which means an ongoing radiotherapy. The diagram 200 of combination therapy can be used to comprehensively analyze the consequence of each treatment modality. It also can be used to evaluate the special monotherapy of radiotherapy combining radio-sensitizer injection. If patient needs a continuing monotherapy, this diagram 200 can continue to draw results on one of coordination systems as previous description of monotherapy.

As an important parameter, the higher OPP % relates to more effective drug/agent/oxygen delivery and oxygenation distribution. Since the result of combination therapy is the comprehensive effect of both treatments, the higher OPP % can be a benefit to both therapy modalities (systemic therapies and local radiation therapy). FIG. 8 demonstrates an ideal case of chemo-radiotherapy for tracking and evaluating during treatment course with a cancer treatment response information diagram 200. It also can be used to evaluate other combination therapies such as immune-radiotherapy, monotherapy such as radiosensitizer-radiotherapy, or any other type of therapy.

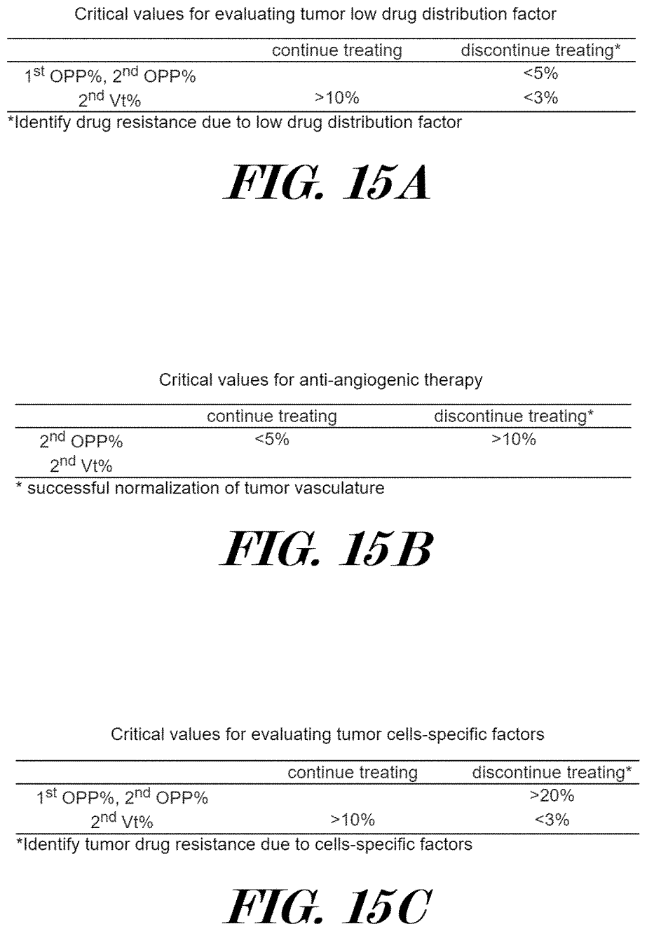

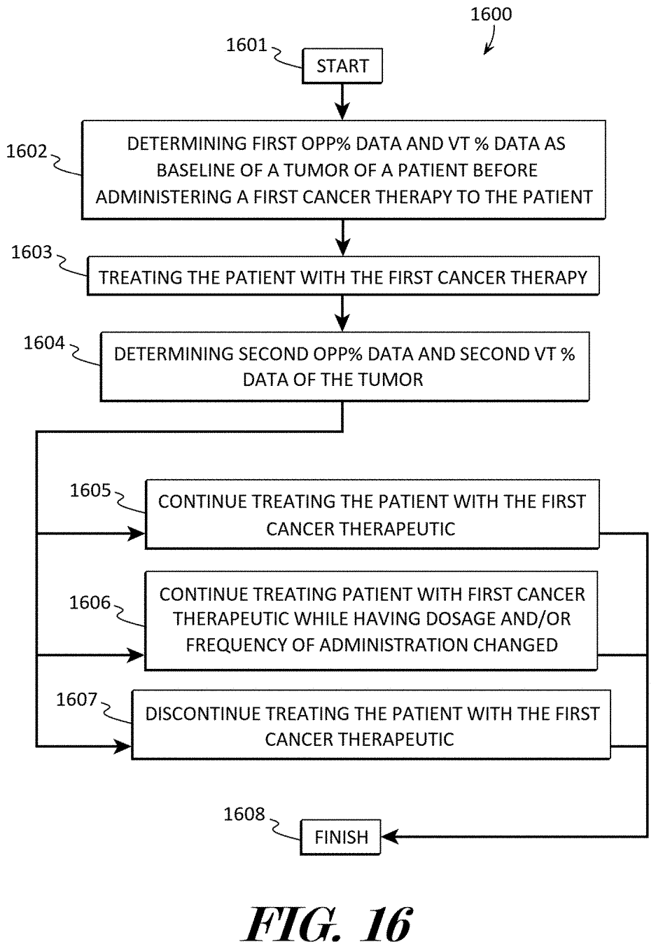

As an example of treatment protocols, and referring to FIGS. 15A-16, anti-angiogenic therapy as one option may be used to treat drug resistance with poor drug distribution. Anti-angiogenic therapies can be used to damage tumor vasculature and alter the hemodynamics and microcirculation inside the tumor, so called normalization of the vasculature for treatment of cancer. However, prescriptions for normalization of the vasculature may depend on the case by case. Different tumors may have different drugs and treatment dose options because any overdose or overtreatment may lead to opposite results.

Although the treatment and dose are precisely designed by clinicians, the key point of this therapeutic strategy in clinical application is to effectively monitor tumor vascular normalization. The present invention can be used to accurately monitor the ability of tumor drug distribution or tumor vascular normalization and evaluate prescription doses in real time, which may be the best choice clinically. In other words, it will be of a unique advantage in monitoring tumor vasculature normalization and treating tumor drug resistance. If treatment resistance is determined to be the cell-specific factors, the therapeutic agent/drug must be replaced.

FIG. 16 illustrates a block diagram of a further example method for precision cancer treatment by identifying drug resistance (“the method”) 1600 according to various embodiments described herein. The method 1600 may start 1601 and a first oxygenated perfusion percentage (OPP %) data and volume change ratio (Vt %) data as baseline of a tumor of a patient before administering a first cancer therapy to the patient may be determined in step 1602.

In step 1603, the patient may be treated with the first cancer therapy.

In step 1604, a second oxygenated perfusion percentage (OPP %) data and a second volume change ratio (Vt %) data of the tumor may be determined.

Next, the method 1600 may proceed to step 1605, step 1606, or step 1607.

In step 1605, the patient may continue to be treated with the first cancer therapeutic so that their therapy is unchanged. After step 1605, the method 1600 may finish 1608.

In step 1606, the patient may continue to be treated with the first cancer therapeutic while having the dosage and/or frequency of administration changed, such as by being increased or decreased. After step 1606, the method 1600 may finish 1608.

In step 1607, the first cancer therapeutic may be discontinued for being administered to the patient. After step 1607, the method 1600 may finish 1608.

Method 1600 Example 1: Low Drug Distribution Leading to Treatment Resistance

In some embodiments, method for precision cancer treatment by identifying drug resistance 1600 may be used to show low drug distribution leading to treatment resistance. In this example, the method 1600 may comprise: determining a first oxygenated perfusion percentage (OPP %) data and a first volume change ratio (Vt %) data as baseline of a tumor of a patient before treatment (step 1602); treating the patient with a first cancer systemic therapy course (step 1603); determining a second oxygenated perfusion percentage (OPP %) data and a second volume change ratio (Vt %) data of the tumor (step 1604); and performing one of: continue treating the patient with the first cancer therapeutic if the second oxygenated perfusion percentage (OPP %) data is substantially equal to the first oxygenated perfusion percentage (OPP %) data and the second volume change ratio (Vt %) data shows greater than 10% shrinkage (step 1605); and discontinue treating the patient with the first cancer therapeutic if the second oxygenated perfusion percentage (OPP %) data and the first oxygenated perfusion percentage (OPP %) data are less than 5% and the second volume change ratio (Vt %) data is not greater than 3% shrinkage (step 1607). Then ineffective treatment is identified as insufficient drug distribution factors and consider anti-angiogenic therapy for normalization of tumor vasculature.

Method 1600 Example 2: Monitoring Normalization of Tumor Vasculature

If a previous cancer therapy or treatment included systemic therapy or radiation therapy, and low drug/oxygen distribution is identified and it may be useful to monitor normalization of the tumor vasculature. In this situation it may only be necessary to check the change of oxygenated perfusion percentage (OPP %) of the tumor. In some embodiments, a method for precision cancer reatment by identifying drug resistance 1600 may be used to monitor normalization of tumor vasculature. In this example, the method 1600 may comprise the steps of: determining a first oxygenated perfusion percentage (OPP %) data and a first volume change ratio (Vt %) data as baseline of a tumor of a patient before treatment (step 1602)—optionally this may be done by inheriting last measurement results of oxygenated perfusion percentage (OPP %) data and volume change ratio (Vt %) data of the tumor; treating the patient with a first anti-angiogenic therapy course (step 1603); determining a second oxygenated perfusion percentage (OPP %) data and volume change ratio (Vt %) data of the tumor (step 1604); and performing one of: continue treating the patient with the anti-angiogenic therapeutic if the second oxygenated perfusion percentage (OPP %) data is less than 5% (step 1605); adjusting the dosage of the first anti-angiogenic therapy course, such as by increasing or decreasing the dosage (step 1606); and discontinue treating the patient with the first anti-angiogenic therapeutic if the second oxygenated perfusion percentage (OPP %) data is higher than 10% (step 1607). It may also be useful for the treating clinician to consider to continue previous systemic therapy or radiation therapy.

Method 1600 Example 3: Drug Resistance is IZdentified (to be the Cells-Specific Factors)

In some embodiments, method for precision cancer treatment by identifying drug resistance 1600 may be used to treat cancer caused by the cells-specific factors. In this example, the method may comprise: determining a first oxygenated perfusion percentage (OPP %) data and volume change ratio (Vt %) data as baseline of a tumor of a patient before treatment (step 1602); treating the patient with a first cancer systemic therapy course (step 1603); determining a second oxygenated perfusion percentage (OPP %) data and volume change ratio (Vt %) data of the tumor (step 1604); and performing one of: continue treating the patient with the first cancer therapeutic if the second oxygenated perfusion percentage (OPP %) data is almost equal to the first oxygenated perfusion percentage (OPP %) data and the second volume change ratio (Vt %) data is greater than 10% shrinkage (step 1605); and discontinue treating the patient with the first cancer therapeutic if the second oxygenated perfusion percentage (OPP %) data and the first oxygenated perfusion percentage (OPP %) data are greater than 20% and the second volume change ratio (Vt %) data is not greater than 3% shrinkage (step 1607). Then ineffective treatment is identified as cells-specific factors and the therapeutic agent/drug must be replaced.

The Cancer Genome Atlas (TCGA) program enables scientists and clinicians to know 10 oncogenic signaling pathways and interpret individuals' genetic codes. Drugs that target these signaling pathways are under development. Currently, there are several drugs that have already been approved by the Food and Drug Administration in the US. These drugs directly target genetic changes in the cells, based on the type, size, and the region of the spread of cancer. The use of drugs to target the changes in the DNA is also known as targeted gene therapy. This precision medicine in cancer treatment is expected to become a mainstream medicine in the near future, a part of it is already in practice.

However, various factors can cause targeted gene mutations and lead to failed targeted therapies. It is reported that drug resistance still exists in targeted therapies. Also, Cancer Genome Atlas (TCGA) program found that 57% of tumors have at least one potentially actionable alteration in their signaling pathways, which means that treatment targeting the genes of these signaling pathways may potentially fail in targeted therapies. Identifying the resistance of cell-specific factors in time will be the first task of clinical application of precision medicine in cancer treatment. The present invention provides the ability to identify the types of drug resistance (especially drug resistance of cells-specific factors), which can greatly improve future precision cancer treatment. In other words, precision medicine is most likely to play a great role in cancer treatment. Similarly, the present invention will also play an important role in improving the efficacy of precision cancer treatment clinically. It will be an indispensable tool for precision medicine in cancer treatment.

With three different examples of the invention, clinicians may have more opportunities to customize cancer treatments on a per-patient basis for achievement of precision medicine in cancer treatment. This will make cancer treatment more controllable and efficient, and ineffective treatment will be greatly reduced.

Referring to FIG. 10, in an exemplary embodiment, a block diagram illustrates a server 3300 which may be used in the system 500, in other systems, or standalone. The server 3300 may be a digital computer that, in terms of hardware architecture, generally includes a processor 3302, input/output (I/O) interfaces 3304, a network interface 3306, a data store 3308, and memory 3310. It should be appreciated by those of ordinary skill in the art that FIG. 11 depicts the server 3300 in an oversimplified manner, and a practical embodiment may include additional components and suitably configured processing logic to support known or conventional operating features that are not described in detail herein. The components (3302, 3304, 3306, 3308, and 3310) are communicatively coupled via a local interface 3312. The local interface 3312 may be, for example but not limited to, one or more buses or other wired or wireless connections, as is known in the art. The local interface 3312 may have additional elements, which are omitted for simplicity, such as controllers, buffers (caches), drivers, repeaters, and receivers, among many others, to enable communications. Further, the local interface 3312 may include address, control, and/or data connections to enable appropriate communications among the aforementioned components.

The processor 3302 is a hardware device for executing software instructions. The processor 3302 may be any custom made or commercially available processor, a central processing unit (CPU), an auxiliary processor among several processors associated with the server 3300, a semiconductor-based microprocessor (in the form of a microchip or chip set), or generally any device for executing software instructions. When the server 3300 is in operation, the processor 3302 is configured to execute software stored within the memory 3310, to communicate data to and from the memory 3310, and to generally control operations of the server 3300 pursuant to the software instructions. The I/O interfaces 3304 may be used to receive user input from and/or for providing system output to one or more devices or components. User input may be provided via, for example, a keyboard, touch pad, and/or a mouse. System output may be provided via a display device and a printer (not shown). I/O interfaces 3304 may include, for example, a serial port, a parallel port, a small computer system interface (SCSI), a serial ATA (SATA), a fibre channel, Infiniband, iSCSI, a PCI Express interface (PCI-x), an infrared (IR) interface, a radio frequency (RF) interface, and/or a universal serial bus (USB) interface.

The network interface 3306 may be used to enable the server 3300 to communicate on a network, such as the Internet, a wide area network (WAN), a local area network (LAN), and the like, etc. The network interface 3306 may include, for example, an Ethernet card or adapter (e.g., 10 BaseT, Fast Ethernet, Gigabit Ethernet, 10 GbE) or a wireless local area network (WLAN) card or adapter (e.g., 802.11a/b/g/n). The network interface 3306 may include address, control, and/or data connections to enable appropriate communications on the network. A data store 3308 may be used to store data. The data store 3308 may include any of volatile memory elements (e.g., random access memory (RAM, such as DRAM, SRAM, SDRAM, and the like)), nonvolatile memory elements (e.g., ROM, hard drive, tape, CDROM, and the like), and combinations thereof. Moreover, the data store 3308 may incorporate electronic, magnetic, optical, and/or other types of storage media. In one example, the data store 3308 may be located internal to the server 3300 such as, for example, an internal hard drive connected to the local interface 3312 in the server 3300. Additionally in another embodiment, the data store 3308 may be located external to the server 3300 such as, for example, an external hard drive connected to the I/O interfaces 3304 (e.g., SCSI or USB connection). In a further embodiment, the data store 3308 may be connected to the server 3300 through a network, such as, for example, a network attached file server.

The memory 3310 may include any of volatile memory elements (e.g., random access memory (RAM, such as DRAM, SRAM, SDRAM, etc.)), nonvolatile memory elements (e.g., ROM, hard drive, tape, CDROM, etc.), and combinations thereof. Moreover, the memory 3310 may incorporate electronic, magnetic, optical, and/or other types of storage media. Note that the memory 3310 may have a distributed architecture, where various components are situated remotely from one another, but can be accessed by the processor 3302. The software in memory 3310 may include one or more software programs, each of which includes an ordered listing of executable instructions for implementing logical functions. The software in the memory 3310 includes a suitable operating system (O/S) 3314 and one or more programs 3316. The operating system 3314 essentially controls the execution of other computer programs, such as the one or more programs 3316, and provides scheduling, input-output control, file and data management, memory management, and communication control and related services. The one or more programs 3316 may be configured to implement the various processes, algorithms, methods, techniques, etc. described herein.

Referring to FIG. 11, in an exemplary embodiment, a block diagram illustrates an electronic device 4400, which may be used in the system 500 or the like. The term “electronic device” as used herein is a type of electronic device comprising circuitry and configured to generally perform functions such as recording audio, photos, and videos; displaying or reproducing audio, photos, and videos; storing, retrieving, or manipulation of electronic data; providing electrical communications and network connectivity; or any other similar function. Non-limiting examples of electronic devices include; personal computers (PCs), workstations, laptops, tablet PCs including the iPad, cell phones including iOS phones made by Apple Inc., Android OS phones, Microsoft OS phones, Blackberry phones, digital music players, or any electronic device capable of running computer software and displaying information to a user, memory cards, other memory storage devices, digital cameras, external battery packs, external charging devices, and the like. Certain types of electronic devices which are portable and easily carried by a person from one location to another may sometimes be referred to as a “portable electronic device” or “portable device”. Some non-limiting examples of portable devices include; cell phones, smart phones, tablet computers, laptop computers, wearable computers such as watches, Google Glasses, etc. and the like.

The electronic device 4400 can be a digital device that, in terms of hardware architecture, generally includes a processor 4402, input/output (I/O) interfaces 4404, a radio 4406, a data store 4408, and memory 4410. It should be appreciated by those of ordinary skill in the art that FIG. 11 depicts the electronic device 4400 in an oversimplified manner, and a practical embodiment may include additional components and suitably configured processing logic to support known or conventional operating features that are not described in detail herein. The components (4402, 4404, 4406, 4408, and 4410) are communicatively coupled via a local interface 4412. The local interface 4412 can be, for example but not limited to, one or more buses or other wired or wireless connections, as is known in the art. The local interface 4412 can have additional elements, which are omitted for simplicity, such as controllers, buffers (caches), drivers, repeaters, and receivers, among many others, to enable communications. Further, the local interface 4412 may include address, control, and/or data connections to enable appropriate communications among the aforementioned components.

The processor 4402 is a hardware device for executing software instructions. The processor 4402 can be any custom made or commercially available processor, a central processing unit (CPU), an auxiliary processor among several processors associated with the electronic device 4400, a semiconductor-based microprocessor (in the form of a microchip or chip set), or generally any device for executing software instructions. When the electronic device 4400 is in operation, the processor 4402 is configured to execute software stored within the memory 4410, to communicate data to and from the memory 4410, and to generally control operations of the electronic device 4400 pursuant to the software instructions. In an exemplary embodiment, the processor 4402 may include a mobile optimized processor such as optimized for power consumption and mobile applications. The I/O interfaces 4404 can be used to receive user input from and/or for providing system output. User input can be provided via, for example, a keypad, a touch screen, a scroll ball, a scroll bar, buttons, bar code scanner, and the like. System output can be provided via a display device such as a liquid crystal display (LCD), touch screen, and the like. The I/O interfaces 4404 can also include, for example, a serial port, a parallel port, a small computer system interface (SCSI), an infrared (IR) interface, a radio frequency (RF) interface, a universal serial bus (USB) interface, and the like. The I/O interfaces 4404 can include a graphical user interface (GUI) that enables a user to interact with the electronic device 4400. Additionally, the I/O interfaces 4404 may further include an imaging device, i.e. camera, video camera, etc.

The radio 4406 enables wireless communication to an external access device or network. Any number of suitable wireless data communication protocols, techniques, or methodologies can be supported by the radio 4406, including, without limitation: RF; IrDA (infrared); Bluetooth; ZigBee (and other variants of the IEEE 802.15 protocol); IEEE 802.11 (any variation); IEEE 802.16 (WiMAX or any other variation); Direct Sequence Spread Spectrum; Frequency Hopping Spread Spectrum; Long Term Evolution (LTE); cellular/wireless/cordless telecommunication protocols (e.g. 3G/4G, or developing 5G etc.); wireless home network communication protocols; paging network protocols; magnetic induction; satellite data communication protocols; wireless hospital or health care facility network protocols such as those operating in the WMTS bands; GPRS; proprietary wireless data communication protocols such as variants of Wireless USB; and any other protocols for wireless communication. The data store 4408 may be used to store data. The data store 4408 may include any of volatile memory elements (e.g., random access memory (RAM, such as DRAM, SRAM, SDRAM, and the like)), nonvolatile memory elements (e.g., ROM, hard drive, tape, CDROM, and the like), and combinations thereof. Moreover, the data store 4408 may incorporate electronic, magnetic, optical, and/or other types of storage media.

The memory 4410 may include any of volatile memory elements (e.g., random access memory (RAM, such as DRAM, SRAM, SDRAM, etc.)), nonvolatile memory elements (e.g., ROM, hard drive, etc.), and combinations thereof. Moreover, the memory 4410 may incorporate electronic, magnetic, optical, and/or other types of storage media. Note that the memory 4410 may have a distributed architecture, where various components are situated remotely from one another, but can be accessed by the processor 4402. The software in memory 4410 can include one or more software programs, each of which includes an ordered listing of executable instructions for implementing logical functions. In the example of FIG. 8, the software in the memory 4410 includes a suitable operating system (O/S) 4414 and programs 4416. The operating system 4414 essentially controls the execution of other computer programs, and provides scheduling, input-output control, file and data management, memory management, and communication control and related services. The programs 4416 may include various applications, add-ons, etc. configured to provide end user functionality with the electronic device 4400. For example, exemplary programs 4416 may include, but not limited to, a web browser, social networking applications, streaming media applications, games, mapping and location applications, electronic mail applications, financial applications, and the like. In a typical example, the end user typically uses one or more of the programs 4416 along with a network.

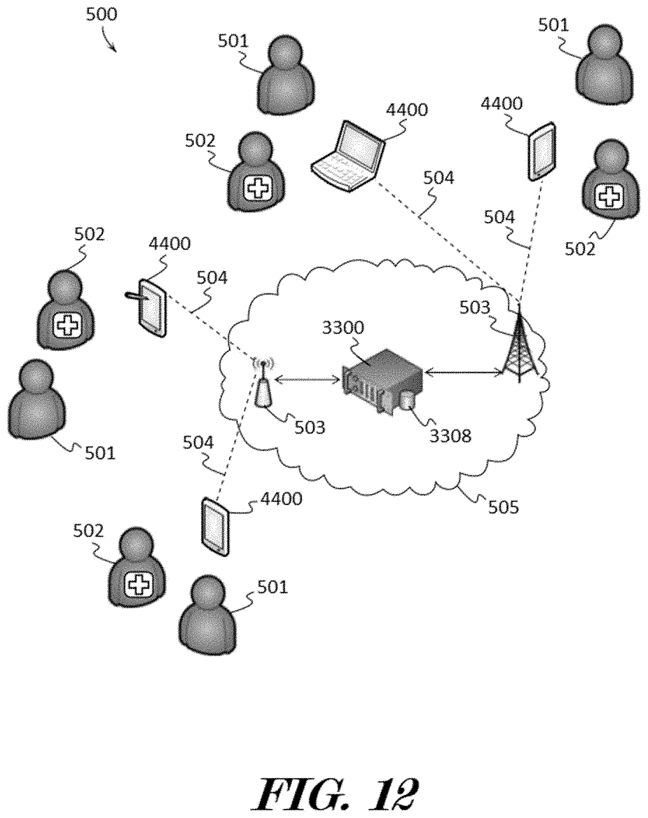

As perhaps best shown by FIG. 12, in some embodiments, as a Therapy-Oriented evaluation tool, a cancer treatment response information diagram 200 can be used as a general platform to share tumor prognostic information between clinicians 502 with different treatment modalities backgrounds to allow for clinician 502 collaboration in optimizing a therapeutic strategy before or during a cancer therapy course of treatment for their patients 501. This collaboration may be performed using a cancer therapy treatment resistance identification system (“the system”) 500. The system 500 may receive the health information of a patient 501, such as one or more diagrams 200, data from one or more diagrams 200, and/or any other data and information related to treatment data, such as sex, age, histopathology, and disease stage, genomic data, treatment plan, which may be stored in a collaboration database 510 and preferably sorted according to treatment site, stage, sex, treatment modality, or any other filtering criteria. Each patient measurement point during a treatment course of a cancer therapy may be collected as therapy response data no matter how effective or ineffective the treatment or therapy is. Based on accumulated and analyzed response data, the system 500 may provide clinicians 502 and patients 501 a quantitative successful probability being calculated by collected similar patient treatment and response data pool and identifying the type of drug resistance for each treatment modality and scheme in order to optimize the therapeutic strategy and achieve precision cancer treatment. In some embodiments, an identification method may include a comparison between treatment effectiveness and patient's quality of life; the possible outcome and side effects and the dose strength of a cancer therapy. In other embodiments, an identification method may include a comparison between the typical rate of tumor response and one or more selected cancer therapies and/or cancer therapy treatment schemes.

An illustrative example of some of the physical components which may comprise a cancer treatment response collaboration system 500 according to some embodiments is presented in FIG. 12. The system 500 is configured to facilitate the transfer of data and information between one or more access points 503, electronic devices 4400, and servers 3300 over a data network 505. Each electronic device 4400 may send data to and receive data from the data network 505 through a network connection 504 with an access point 503. A data store 3308 accessible by the server 3300 may contain one or more databases. The data may comprise any information pertinent to one or more patients 501, clinicians 502, and/or other users which may be input into the system 500 including information on or describing cancer therapy data of one or more patients 501, information requested by one or more clinicians 502, information supplied by one or more clinicians 502, and any other information which a clinician 502 may use for cancer treatment evaluation and collaboration of one or more patients 501.

In this example, the system 500 comprises at least one electronic device 4400 (but preferably more than two electronic devices 4400) configured to be operated by one or more clinicians 502. In some embodiments, the system 500 may be configured to facilitate the communication of information between one or more clinicians 502, through their respective electronic devices 4400 and/or servers 3300 of the system 500. Electronic devices 4400 can be mobile devices, such as laptops, tablet computers, personal digital assistants, smart phones, and the like, that are equipped with a wired or wireless network interface capable of sending data to one or more servers 3300 with access to one or more data stores 3308 over a network 505 such as a wired local area network or wireless local area network. Additionally, user electronic devices 4400 can be fixed devices, such as desktops, imagining devices, medical workstations, treatment and administration workstations, and the like, that are equipped with a wireless or wired network interface capable of sending data to one or more servers 3300 with access to one or more data stores 3308 over a wireless or wired local area network 505. The present invention may be implemented on at least one electronic device 4400 and/or server 3300 programmed to perform one or more of the steps described herein. In some embodiments, more than one user electronic device 4400 and/or server 3300 may be used, with each being programmed to carry out one or more steps of a method or process described herein.

Referring now to FIG. 13, a block diagram showing some software rules engines which may be found in a system 500 (FIG. 12) which may optionally be configured to run on a server 3300 (FIGS. 10 and 12) and an example of a collaboration database 510 according to various embodiments described herein are illustrated, respectively. In some embodiments, one or more servers 3300 may be configured to run one or more software rules engines or programs such as a communication application 511, association application 512, and/or an estimation application 513. In this embodiment, the applications 511, 512, 513, are configured to run on at least one server 3300. The server 3300 may be in electronic communication with a data store 3308 comprising a database, such as a collaboration database 510. The engines 511, 512, 513, may read, write, or otherwise access data in one or more databases of the data store 308. Additionally, data may be sent and received to and from one or more electronic devices 4400 (FIGS. 11 and 12) which may be in wired and/or wireless electronic communication with the server 3300 through a network 505. In other embodiments, a communication application 511, association application 512, and/or an estimation application 513 may be configured to run on a electronic device 4400 and/or server 3300 with data transferred to and from one or more servers 3300 in communication with a data store 3308 through a network 505. In still further embodiments, a server 3300 or an electronic device 4400 may be configured to run a communication application 511, association application 512, and/or an estimation application 513.

In some embodiments, the system 500 may comprise a database, such as a collaboration database 510, optionally stored on a data store 3308 accessible to a communication application 511, association application 512, and/or an estimation application 513. In further embodiments, a collaboration database 510 may be stored on a data store 4408 of an electronic device 4400. A collaboration database 510 may comprise any data and information pertinent to one or more patients 501 and/or clinicians 502 of the system 500. This data may include information which may describe the cancer therapy, results of cancer therapy, and other health information which may describe a patient 501. For example, this health information may include oxygenated perfusion percentage data OPP %, volume change ratio Vt % data, imaging data, types of cancer therapies received, durations of cancer therapies received, doses of cancer therapies received, or any other health information which may describe one or more patients 501 of a clinician 502. Additionally, the data of two or more patients 501 and/or clinicians 502 may be pooled so that the all the information which may describe the cancer therapy, results of cancer therapy, and other health information of all of the patients 501 in the collaboration database 510 may be searched.

The communication application 511 may comprise a computer program which may be executed by a computing device processor, such as a processor 3302 (FIG. 10) and/or a processor 4402 (FIG. 11), and which may be configured to govern electronic communication between severs 3300 and electronic devices 4400. Data from severs 3300 and electronic devices 4400 may be received by the communication application 511 which may then electronically communicate the data to the association application 512 and estimation application 513. Likewise, data from the association application 512 and estimation application 513 may be received by the communication application 511 which may then electronically communicate the data to servers 3300 and electronic devices 4400. In some embodiments, the communication application 511 may govern the electronic communication by initiating, maintaining, reestablishing, and terminating electronic communication between one or more electronic devices 4400 and servers 3300. In further embodiments, the communication application 511 may control the network interface 3306 (FIG. 10) of the server 3300 to send and receive data to and from one or more electronic devices 4400 and other servers 3300 through a network connection 504 (FIG. 12) over a network 505 (FIG. 12).

The association application 512 may comprise a computer program which may be executed by a computing device processor, such as a processor 3302 (FIG. 10) and/or a processor 4402 (FIG. 11), and which may be configured to store, retrieve, modify, create, and/or delete data and information which may describe the cancer therapy, results of cancer therapy, and other health information of a patient 501, including oxygenated perfusion percentage data OPP %, volume change ratio Vt % data, imaging data, types of cancer therapies received, durations of cancer therapies received, doses of cancer therapies received, or any other health information which may describe one or more patients 501 of a clinician 502 into and from the collaboration database 510. In some embodiments, the association application 512 receive data from the communication application 511 and/or estimation application 513 and associate the data with information which may describe the cancer therapy, results of cancer therapy, and other health information of a patient 501, including oxygenated perfusion percentage data OPP %, volume change ratio Vt % data, imaging data, types of cancer therapies received, durations of cancer therapies received, doses of cancer therapies received, or any other health information which may describe one or more patients 501 of a clinician 502 into the collaboration database 510. In further embodiments, the association application 512 retrieve data from the collaboration database 510, such as information which may describe the cancer therapy, results of cancer therapy, and other health information of a patient 501, including oxygenated perfusion percentage data OPP %, volume change ratio Vt % data, imaging data, types of cancer therapies received, durations of cancer therapies received, doses of cancer therapies received, or any other health information which may describe one or more patients 501 of a clinician 502, and send or communicate the data to the communication application 511 and/or estimation application 513.

The estimation application 513 may comprise a computer program which may be executed by a computing device processor, such as a processor 3302 (FIG. 10) and/or a processor 4402 (FIG. 11), and which may be configured to compare data received from the communication application 511 to data received from the association application 512. In some embodiments, the estimation application 513 may compare the health information of a particular patient 501 received by the communication application 511 through the electronic device 4400 of a clinician 502 to the health information of one or more patients 501, including the pooled health information and data of all the patients 501 in the collaboration database 510, retrieved by the association application 512 from the collaboration database 510. The estimation application 513 may be configured to identify the type of drug resistance of how the cancer tumor of the particular patient 501 would respond to a cancer therapy that the particular patient 501 has not yet received based upon the oxygenated perfusion percentage data OPP % and volume change ratio Vt % pooled data of the identified one or patients in the collaboration database 510 that did undergo the cancer therapy that the particular patient has not yet received. Based on the pooled and analyzed response data, the estimation application 513 of the system 500 may provide clinicians 502 and patients 501 a quantitative identification method for each treatment modality and scheme in order to optimize the therapeutic strategy and achieve precision cancer treatment.

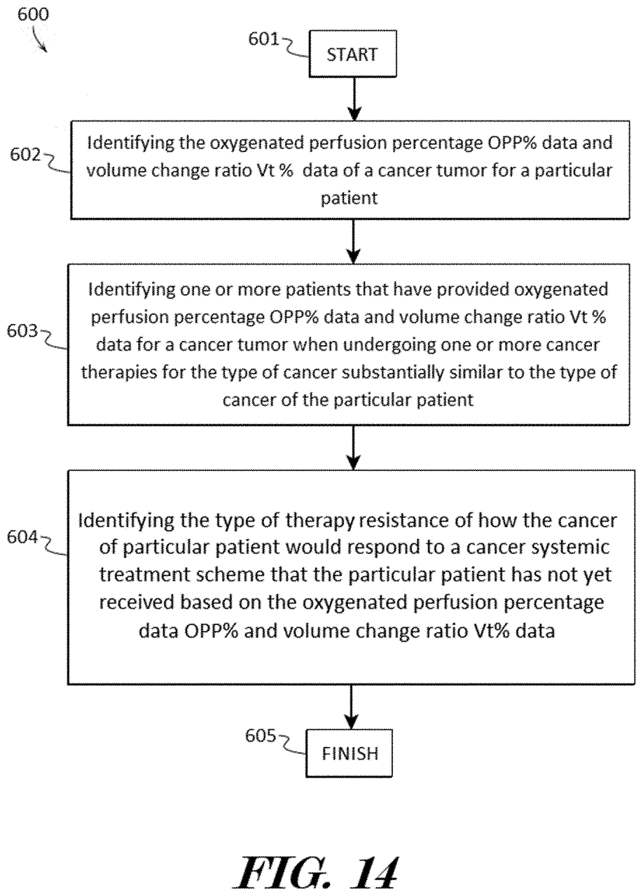

FIG. 14 shows a block diagram of an example of a computer-implemented method for precision cancer treatment by identifying drug resistance (“the method”) 600 which may utilize one or more cancer treatment response information diagrams 200 and a cancer therapy treatment resistance identification system 500 according to various embodiments described herein. In some embodiments, the method 600 may be used to provide clinicians 502 and patients 501 a quantitative identification method for each cancer therapy treatment modality or treatment scheme in order to optimize the therapeutic strategy and achieve precision cancer treatment using one or more electronic devices 4400 and/or servers 3300. One or more steps of the method 600 may be performed by a communication application 511, an association application 512, and/or an estimation application 513 which may be executed by the processor of an electronic device, such as a processor 3302 (FIG. 10) and/or a processor 4402 (FIG. 11). In some embodiments, the method 600 may be used for the treatment of human solid tumors, although in further embodiments, the method 600 may be used for the treatment of solid tumors in any mammal or other organism.

In some embodiments, the method 600 may start 601 and the oxygenated perfusion percentage data OPP % and volume change ratio Vt % data of a cancer tumor for a particular patient 501 (FIG. 12) may be identified in step 602. In further embodiments, step 602 may be performed using steps 110-115 of the cancer drug resistance identification method 100 of FIG. 1. In still further embodiments, step 118 and/or 119 of the cancer drug resistance identification method 100 of FIG. 1 may also be performed in step 602. This data may be communicated by a communication application 511 (FIG. 13) and an association application 512 (FIG. 13) to a collaboration database 510 (FIG. 13).

Next, in step 603 one or more patients 501 that have provided oxygenated perfusion percentage data OPP % and volume change ratio Vt % data, such as by one or more steps of the cancer drug resistance identification method 100 of FIG. 1, for a cancer tumor when undergoing one or more cancer therapies for the type of cancer substantially similar to the type of cancer of the particular patient 501 may be identified in the collaboration database 510 by the association application 512. Preferably, the association application 512 may retrieve this data without retrieving any personally identifying information of the one or more patients 501.

In step 604, an identification method of how the cancer tumor of the particular patient 501 would respond to a cancer therapy treatment scheme that the particular patient 501 has not yet received may be generated by the estimation application 513 based upon the oxygenated perfusion percentage data OPP % and volume change ratio Vt % pooled data in the collaboration database 510 of the identified one or patients 501 that did undergo the cancer therapy treatment scheme that the particular patient 501 has not yet received. In some embodiments, the identification method may include how the percentage of tumor complete response and partial response for each particular therapeutic modality. In further embodiments, an identification method may include a comparison between treatment effectiveness and patient's quality of life; the possible outcome and the side effects and the dose strength of a cancer therapy. In other embodiments, an identification method may include a comparison between the typical rate of tumor response and one or more selected cancer therapies and/or cancer therapy treatment schemes. An identification method may be generated for each cancer therapy that has been administered to one or more patients having health information, such as oxygenated perfusion percentage data OPP % and volume change ratio Vt % data, for a substantially similar type of cancer as the particular patient 501. After step 604, the method 600 may finish 605.

It will be appreciated that some exemplary embodiments described herein may include one or more generic or specialized processors (or “processing devices”) such as microprocessors, digital signal processors, customized processors and field programmable gate arrays (FPGAs) and unique stored program instructions (including both software and firmware) that control the one or more processors to implement, in conjunction with certain non-processor circuits, some, most, or all of the functions of the methods and/or systems described herein. Alternatively, some or all functions may be implemented by a state machine that has no stored program instructions, or in one or more application specific integrated circuits (ASICs), in which each function or some combinations of certain of the functions are implemented as custom logic. Of course, a combination of the two approaches may be used. Moreover, some exemplary embodiments may be implemented as a computer-readable storage medium having computer readable code stored thereon for programming a computer, server, appliance, device, etc. each of which may include a processor to perform methods as described and claimed herein. Examples of such computer-readable storage mediums include, but are not limited to, a hard disk, an optical storage device, a magnetic storage device, a ROM (Read Only Memory), a PROM (Programmable Read Only Memory), an EPROM (Erasable Programmable Read Only Memory), an EEPROM (Electrically Erasable Programmable Read Only Memory), a Flash memory, and the like.

Embodiments of the subject matter and the functional operations described in this specification can be implemented in digital electronic circuitry, or in computer software, firmware, or hardware, including the structures disclosed in this specification and their structural equivalents, or in combinations of one or more of them. Embodiments of the subject matter described in this specification can be implemented as one or more computer program products, i.e., one or more modules of computer program instructions encoded on a tangible program carrier for execution by, or to control the operation of, data processing apparatus. The tangible program carrier can be a propagated signal or a computer readable medium. The propagated signal is an artificially generated signal, e.g., a machine generated electrical, optical, or electromagnetic signal that is generated to encode information for transmission to suitable receiver apparatus for execution by a computer. The computer readable medium can be a machine readable storage device, a machine readable storage substrate, a memory device, a composition of matter effecting a machine readable propagated signal, or a combination of one or more of them.

A computer program (also known as a program, software, software application, application, script, or code) can be written in any form of programming language, including compiled or interpreted languages, or declarative or procedural languages, and it can be deployed in any form, including as a standalone program or as a module, component, subroutine, or other unit suitable for use in a computing environment. A computer program does not necessarily correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, sub programs, or portions of code). A computer program can be deployed to be executed on one computer or on multiple computers that are located at one site or distributed across multiple sites and interconnected by a communication network.

Additionally, the logic flows and structure block diagrams described in this patent document, which describe particular methods and/or corresponding acts in support of steps and corresponding functions in support of disclosed structural means, may also be utilized to implement corresponding software structures and algorithms, and equivalents thereof. The processes and logic flows described in this specification can be performed by one or more programmable processors (computing device processors) executing one or more computer applications or programs to perform functions by operating on input data and generating output.

Processors suitable for the execution of a computer program include, by way of example, both general and special purpose microprocessors, and any one or more processors of any kind of digital computer. Generally, a processor will receive instructions and data from a read only memory or a random access memory or both. The essential elements of a computer are a processor for performing instructions and one or more memory devices for storing instructions and data. Generally, a computer will also include, or be operatively coupled to receive data from or transfer data to, or both, one or more mass storage devices for storing data, e.g., magnetic, magneto optical disks, solid state drives, or optical disks. However, a computer need not have such devices.

Computer readable media suitable for storing computer program instructions and data include all forms of non volatile memory, media and memory devices, including by way of example semiconductor memory devices, e.g., EPROM, EEPROM, and flash memory devices; magnetic disks, e.g., internal hard disks or removable disks; magneto optical disks; and CD ROM and DVD ROM disks. The processor and the memory can be supplemented by, or incorporated in, special purpose logic circuitry.

To provide for interaction with a user, embodiments of the subject matter described in this specification can be implemented on a computer having a display device, e.g., a CRT (cathode ray tube) or LCD (liquid crystal display) monitor, for displaying information to the user and a keyboard and a pointing device, e.g., a mouse or a trackball, by which the user can provide input to the computer. Other kinds of devices can be used to provide for interaction with a user as well; for example, feedback provided to the user can be any form of sensory feedback, e.g., visual feedback, auditory feedback, or tactile feedback; and input from the user can be received in any form, including acoustic, speech, or tactile input.

Embodiments of the subject matter described in this specification can be implemented in a computing system that includes a back end component, e.g., as a data server, or that includes a middleware component, e.g., an application server, or that includes a front end component, e.g., a client computer having a graphical user interface or a Web browser through which a user can interact with an implementation of the subject matter described is this specification, or any combination of one or more such back end, middleware, or front end components. The components of the system can be interconnected by any form or medium of digital data communication, e.g., a communication network such as PACS system. Examples of communication networks include a local area network (“LAN”) and a wide area network (“WAN”), e.g., the Internet.

The computing system can include clients and servers. A client and server are generally remote from each other and typically interact through a communication network or the cloud. The relationship of client and server arises by virtue of computer programs running on the respective computers and having a client server relationship to each other.

Further, many embodiments are described in terms of sequences of actions to be performed by, for example, elements of a computing device. It will be recognized that various actions described herein can be performed by specific circuits (e.g., application specific integrated circuits (ASICs)), by program instructions being executed by one or more processors, or by a combination of both. Additionally, these sequence of actions described herein can be considered to be embodied entirely within any form of computer readable storage medium having stored therein a corresponding set of computer instructions that upon execution would cause an associated processor to perform the functionality described herein. Thus, the various aspects of the invention may be embodied in a number of different forms, all of which have been contemplated to be within the scope of the claimed subject matter. In addition, for each of the embodiments described herein, the corresponding form of any such embodiments may be described herein as, for example, “logic configured to” perform the described action.

The computer system may also include a main memory, such as a random access memory (RAM) or other dynamic storage device (e.g., dynamic RAM (DRAM), static RAM (SRAM), and synchronous DRAM (SDRAM)), coupled to the bus for storing information and instructions to be executed by processor. In addition, the main memory may be used for storing temporary variables or other intermediate information during the execution of instructions by the processor. The computer system may further include a read only memory (ROM) or other static storage device (e.g., programmable ROM (PROM), erasable PROM (EPROM), and electrically erasable PROM (EEPROM)) coupled to the bus for storing static information and instructions for the processor.

The computer system may also include a disk controller coupled to the bus to control one or more storage devices for storing information and instructions, such as a magnetic hard disk, and a removable media drive (e.g., floppy disk drive, read-only compact disc drive, read/write compact disc drive, compact disc jukebox, tape drive, and removable magneto-optical drive). The storage devices may be added to the computer system using an appropriate device interface (e.g., small computer system interface (SCSI), integrated device electronics (IDE), enhanced-IDE (E-IDE), direct memory access (DMA), or ultra-DMA).

The computer system may also include special purpose logic devices (e.g., application specific integrated circuits (ASICs)) or configurable logic devices (e.g., simple programmable logic devices (SPLDs), complex programmable logic devices (CPLDs), and field programmable gate arrays (FPGAs)).

The computer system may also include a display controller coupled to the bus to control a display, such as a cathode ray tube (CRT), liquid crystal display (LCD) or any other type of display, for displaying information to a computer user. The computer system may also include input devices, such as a keyboard and a pointing device, for interacting with a computer user and providing information to the processor. Additionally, a touch screen could be employed in conjunction with display. The pointing device, for example, may be a mouse, a trackball, or a pointing stick for communicating direction information and command selections to the processor and for controlling cursor movement on the display. In addition, a printer may provide printed listings of data stored and/or generated by the computer system.

The computer system performs a portion or all of the processing steps of the invention in response to the processor executing one or more sequences of one or more instructions contained in a memory, such as the main memory. Such instructions may be read into the main memory from another computer readable medium, such as a hard disk or a removable media drive. One or more processors in a multi-processing arrangement may also be employed to execute the sequences of instructions contained in main memory. In alternative embodiments, hard-wired circuitry may be used in place of or in combination with software instructions. Thus, embodiments are not limited to any specific combination of hardware circuitry and software.

As stated above, the computer system includes at least one computer readable medium or memory for holding instructions programmed according to the teachings of the invention and for containing data structures, tables, records, or other data described herein. Examples of computer readable media are compact discs, hard disks, floppy disks, tape, magneto-optical disks, PROMs (EPROM, EEPROM, flash EPROM), DRAM, SRAM, SDRAM, or any other magnetic medium, compact discs (e.g., CD-ROM), or any other optical medium, punch cards, paper tape, or other physical medium with patterns of holes, a carrier wave (described below), or any other medium from which a computer can read.

Stored on any one or on a combination of computer readable media, the present invention includes software for controlling the computer system, for driving a device or devices for implementing the invention, and for enabling the computer system to interact with a human user. Such software may include, but is not limited to, device drivers, operating systems, development tools, and applications software. Such computer readable media further includes the computer program product of the present invention for performing all or a portion (if processing is distributed) of the processing performed in implementing the invention.

The computer code or software code of the present invention may be any interpretable or executable code mechanism, including but not limited to scripts, interpretable programs, dynamic link libraries (DLLs), Java classes, and complete executable programs. Moreover, parts of the processing of the present invention may be distributed for better performance, reliability, and/or cost.

Various forms of computer readable media may be involved in carrying out one or more sequences of one or more instructions to processor for execution. For example, the instructions may initially be carried on a magnetic disk of a remote computer. The remote computer can load the instructions for implementing all or a portion of the present invention remotely into a dynamic memory and send the instructions over the air (e.g. through a wireless cellular network or WiFi network). A modem local to the computer system may receive the data over the air and use an infrared transmitter to convert the data to an infrared signal. An infrared detector coupled to the bus can receive the data carried in the infrared signal and place the data on the bus. The bus carries the data to the main memory, from which the processor retrieves and executes the instructions. The instructions received by the main memory may optionally be stored on storage device either before or after execution by processor.

The computer system also includes a communication interface coupled to the bus. The communication interface provides a two-way data communication coupling to a network link that is connected to, for example, a local area network (LAN), or to another communications network such as the Internet. For example, the communication interface may be a network interface card to attach to any packet switched LAN. As another example, the communication interface may be an asymmetrical digital subscriber line (ADSL) card, an integrated services digital network (ISDN) card or a modem to provide a data communication connection to a corresponding type of communications line. Wireless links may also be implemented. In any such implementation, the communication interface sends and receives electrical, electromagnetic or optical signals that carry digital data streams representing various types of information.

The network link typically provides data communication to the cloud through one or more networks to other data devices. For example, the network link may provide a connection to another computer or remotely located presentation device through a local network (e.g., a LAN) or through equipment operated by a service provider, which provides communication services through a communications network. In preferred embodiments, the local network and the communications network preferably use electrical, electromagnetic, or optical signals that carry digital data streams. The signals through the various networks and the signals on the network link and through the communication interface, which carry the digital data to and from the computer system, are exemplary forms of carrier waves transporting the information. The computer system can transmit and receive data, including program code, through the network(s) and, the network link and the communication interface. Moreover, the network link may provide a connection through a LAN to a user device or client device such as a personal digital assistant (PDA), laptop computer, tablet computer, smartphone, or cellular telephone. The LAN communications network and the other communications networks such as cellular wireless and wifi networks may use electrical, electromagnetic or optical signals that carry digital data streams. The processor system can transmit notifications and receive data, including program code, through the network(s), the network link and the communication interface.

Although the present invention has been illustrated and described herein with reference to preferred embodiments and specific examples thereof, it will be readily apparent to those of ordinary skill in the art that other embodiments and examples may perform similar functions and/or achieve like results. All such equivalent embodiments and examples are within the spirit and scope of the present invention, are contemplated thereby, and are intended to be covered by the following claims.

What is claimed is:

1. A method for precision cancer treatment by identifying drug resistance in a first cancer therapy for a particular patient implemented by an electronic device comprising a processor, a data input/output device, and a display input/output device in which data is visualized on a cancer treatment response information diagram wherein the diagram comprises two independent symmetrical coordination systems as a triangle structure having a poor oxygenated perfusion apex, a first well oxygenated perfusion apex, a second well oxygenated perfusion apex, a first change in tumor volume coordinate graph extending from the poor oxygenated perfusion apex and the first well oxygenated perfusion apex, and a second change in tumor volume coordinate graph extending from the poor oxygenated perfusion apex and the second well oxygenated perfusion apex, and wherein the method comprises the steps of:
a. acquiring tumor baseline data of the particular patient generated by dynamic contrast enhanced T2-weighted MR imaging technique with a data input/output device;
b. acquiring tumor enhanced data of the particular patient with increasing body blood oxyhemoglobin (HbO₂) concentration, which is generated by same dynamic contrast enhanced T2-weighted MR imaging technique, with a data input/output device;
c. calculating tumor volume based on acquired tumor T2-weighted MR imaging data with the processor;
d. calculating a tumor volume change ratio (Vt %) data with the processor;
e. calculating tumor voxel's enhanced signal intensity (ΔSI) data with the processor;
f. calculating tumor oxygenated perfusion percentage (OPP %) data with the processor;
g. calculating different thresholds of oxygenated perfusion percentage (OPP %) data and maps with the processor;
h. creating special threshold maps with the processor;
i. plotting OPP % data and Vt % data of the particular patient on the treatment response information diagram with the processor on the display input/output device; and
j. identifying a type of drug resistance based on analyzing the cancer treatment response information diagram.

2. The method of claim 1, wherein the oxygenated perfusion percentage data (OPP %) uses a threshold technique in processing dynamic contrast enhancement T2 weighted MM data for quantitatively measuring patient tumor microcirculation from one of the following: during the first cancer therapy; and before the first cancer therapy.

3. The method of claim 1, wherein the method further comprises integrating tumor volume change information (volume change ratio Vt %) and tumor microcirculation information (OPP %) into one therapeutic response information point on the cancer treatment response information diagram for evaluation treatment response.

4. The method of claim 1, wherein the method further comprises plotting the oxygenated perfusion percentage (OPP %) data and volume change ratio (Vt %) data obtained before the first cancer therapy and plotting the oxygenated perfusion percentage (OPP %) data and volume change ratio (Vt %) data obtained during the first cancer therapy on the cancer treatment response information diagram.

5. The method of claim 1, wherein the oxygenated perfusion percentage (OPP %) data and volume change ratio (Vt %) data obtained during the first cancer therapy for the particular patient is displayed or plotted on the first change in tumor volume coordinate graph extending from the poor oxygenated perfusion apex and the first well oxygenated perfusion apex of the cancer treatment response information diagram, and wherein the oxygenated perfusion percentage (OPP %) data and volume change ratio (Vt %) data obtained during a second cancer therapy for the particular patient is plotted on the second change in tumor volume coordinate graph extending from the poor oxygenated perfusion apex and the second well oxygenated perfusion apex of the cancer treatment response information diagram.

6. The method of claim 5, wherein oxygenated perfusion percentage (OPP %) data and volume change ratio (Vt %) data from at least two treatment course time points are plotted on the cancer treatment response information diagram, wherein if the treatment response information is consistent with a at least two continuous measurements during treatment, these response information points are used to identify the type of tumor resistance in systemic therapies selected from one or the following: the drug resistance of a low drug distribution factor is identified by a low oxygen perfusion percentage (OPP %) and a small volume change rate (Vt %); and the resistance of cell-specific factors is determined by a high oxygen perfusion percentage (OPP %) and a small volume change ratio (Vt %), wherein identification of normalizing tumor vasculature treatment to treat drug resistance of low drug distribution follows a successful anti-angiogenic therapy for normalization tumor vasculature and is identified by the oxygen perfusion percentage OPP % being significant increased.

7. The method of claim 5, wherein the first cancer therapy is selected from the group consisting essentially of: systemic therapies (chemotherapy, molecular targeted therapy, immunotherapy, gene therapy, photodynamic therapy), local irradiation therapies (radiotherapy, hyperthermia therapy), and systemic therapies-local irradiation therapies combinations.

8. The method of claim 5, wherein the second cancer therapy is selected from the group consisting essentially of: systemic therapies (chemotherapy, molecular targeted therapy, immunotherapy, gene therapy, photodynamic therapy), local irradiation therapies (radiation therapy, hyperthermia therapy), systemic therapies-local irradiation therapies combinations.

9. The method of claim 1, wherein the method comprises the construction of cancer treatment response information diagram. wherein the diagram comprises two independent symmetrical coordination systems as a triangle structure having a poor oxygenated perfusion apex, a first well oxygenated perfusion apex, a second well oxygenated perfusion apex, a first change in tumor volume coordinate graph extending from the poor oxygenated perfusion apex and the first well oxygenated perfusion apex, and a second change in tumor volume coordinate graph extending from the poor oxygenated perfusion apex and the second well oxygenated perfusion apex.

10. A method for precision cancer treatment by identifying drug resistance by identifying a type of drug resistance of how a cancer tumor of a particular patient would respond to a cancer therapy the particular patient has not yet received for achieving evidence-based precision medicine, the method comprising:
a. identifying an oxygenated perfusion percentage (OPP %) data and a volume change ratio (Vt %) data of a cancer tumor for the particular patient;
b. identifying one or more other patients that have provided oxygenated perfusion percentage OPP % data, volume change ratio Vt % data and treatment schemes for their cancer tumor when undergoing one or more cancer therapies for a type of cancer substantially similar to the type of cancer of the particular patient; and
c. identifying the type of drug resistance of how the cancer tumor of the particular patient would respond to a cancer therapy that the particular patient has not yet received based upon the oxygenated perfusion percentage data OPP % and volume change ratio Vt % data,
d. wherein the method is performed by one or more electronic devices.

11. The method of claim 11, wherein the oxygenated perfusion percentage data (OPP %) and volume change ratio (Vt %) data is visualized on a cancer treatment response information diagram, wherein the diagram comprises two independent symmetrical coordination systems as a triangle structure having a poor oxygenated perfusion apex, a first well oxygenated perfusion apex, a second well oxygenated perfusion apex, a first change in tumor volume coordinate graph extending from the poor oxygenated perfusion apex and the first well oxygenated perfusion apex, and a second change in tumor volume coordinate graph extending from the poor oxygenated perfusion apex and the second well oxygenated perfusion apex.

12. The method of claim 11, wherein the method further comprises displaying a reconstruction tumor oxygenated perfusion percentage OPP % pseudo color image during a course of the cancer treatment on a display of an electronic device.

13. The method of claim 12, wherein the method further comprises plotting the oxygenated perfusion percentage data OPP % and volume change ratio Vt % data obtained before the cancer therapy and plotting the oxygenated perfusion percentage data OPP % and volume change ratio Vt % data obtained during the cancer therapy on the treatment response information diagram.

14. The method of claim 12, wherein the method further comprises plotting the oxygenated perfusion percentage data OPP % and volume change ratio Vt % data obtained before and during the cancer therapy on the treatment response information diagram to identify the type of drug resistance in systemic therapies.

15. The method of claim 12, wherein the method further comprises plotting the oxygenated perfusion percentage data OPP % and a Reconstruction OPP % map obtained during a cancer radiation treatment course to determine where tumor low oxygenation regions are targeted for a Biologically-Guided Radiation Therapy.

16. The method of claim 12, wherein the oxygenated perfusion percentage OPP % data and volume change ratio Vt % data obtained during a first cancer therapy for the particular patient is plotted on the first change in tumor volume coordinate graph extending from the poor oxygenated perfusion apex and the first well oxygenated perfusion apex of the cancer treatment response information diagram, and wherein the oxygenated perfusion percentage OPP % data and volume change ratio Vt % data obtained during a second cancer therapy for the particular patient is plotted on the second change in tumor volume coordinate graph extending from the poor oxygenated perfusion apex and the second well oxygenated perfusion apex of the cancer treatment response information diagram.

17. A method for precision cancer treatment by identifying drug resistance, the method comprising:
a. determining a first oxygenated perfusion percentage (OPP %) data and volume change ratio (Vt %) data as baseline of a tumor of a patient before administering a first cancer therapy to the patient;
b. treating the patient with the first cancer therapy; and
c. determining a second oxygenated perfusion percentage (OPP %) data and a second volume change ratio (Vt %) data of the tumor.

18. The method of claim 17, wherein the first cancer therapy is a systemic therapy, and further comprising the step of performing one of: continue treating the particular patient with the first cancer therapeutic if the second oxygenated perfusion percentage (OPP %) data is approximately equal to the first oxygenated perfusion percentage (OPP %) data and the second volume change ratio (Vt %) data shows greater than 10 percent shrinkage;
and discontinue treating the particular patient with the first cancer therapeutic if the second oxygenated perfusion percentage (OPP %) data and the first oxygenated perfusion percentage (OPP %) data are less than 5 percent and the second volume change ratio (Vt %) data is not greater than 3 percent shrinkage.

19. The method of claim 17, wherein the first cancer therapy is an anti-angiogenic therapy, and further comprising the step of performing one of: continue treating the particular patient with the first cancer therapeutic if the second oxygenated perfusion percentage (OPP %) data is less than 3 percent; increase dosage of the first cancer therapy; and discontinue treating the particular patient with the first cancer therapeutic if the second oxygenated perfusion percentage (OPP %) data is higher than 10 percent.

20. The method of claim 17, wherein the first cancer therapy is a systemic therapy, and further comprising the step of performing one of: continue treating the particular patient with the first cancer therapeutic if the second oxygenated perfusion percentage (OPP %) data is approximately equal to the first oxygenated perfusion percentage (OPP %) data and the second volume change ratio (Vt %) data shows greater than 10% shrinkage; and increase dosage of the first cancer therapy; and discontinue treating the particular patient with the first cancer therapeutic if the second oxygenated perfusion percentage (OPP %) data and the first oxygenated perfusion percentage (OPP %) data are greater than 20 percent and the second volume change ratio (Vt %) data is not greater than 3 percent shrinkage.
